(19) United States
(12) Patent Application Publication
(10) Pub. No.: US 2026/0076982 A1
(43) Pub. Date: Mar. 19, 2026

(54) Prodrugs of Cannbidiol [CBD]-Type Phytocannabinoids and a Process for Preparation Thereof

(71) Applicant: Council of Scientific and Industrial Research, New Delhi (IN)

(72) Inventors: Pankaj Singh CHAM, Jammu (IN); Pankul KOTWAL, Jammu (IN); Kuhu SHARMA, Jammu (IN); Sukhleen KAUR, Jammu (IN); Durga Prasad MINDALA, Jammu (IN); Ajay KUMAR, Jammu (IN); Utpal NANDI, Jammu (IN); Parvinder PAL SINGH, Jammu (IN)

(21) Appl. No.: 18/992,444

(22) PCT Filed: Jul. 6, 2023
(86) PCT No.: PCT/IN2023/050656

§ 371 Date: Jan. 8, 2025

(30) Foreign Application Priority Data
Jul. 8, 2022 (IN) 202211039597

(51) Int. Cl. A61K 31/00; A61K 47/10; A61K 47/20; A61K 47/44; C07D 211/14; C07D 211/46; C07D 295/15

(52) U.S. Cl. CPC A61K 31/658; A61K 47/10; A61K 47/20; A61K 47/44; C07D 211/14; C07D 211/46; C07D 295/15

(57) ABSTRACT

The present invention provides the novel prodrugs of cannabidio-type phytocannabinoids having the general Formula A and its process thereof, where one or both the hydroxyl groups are attached to the other counter parts through ester bond. The present invention also studies the drug release studies in Ex-vivo and In-vivo systems.

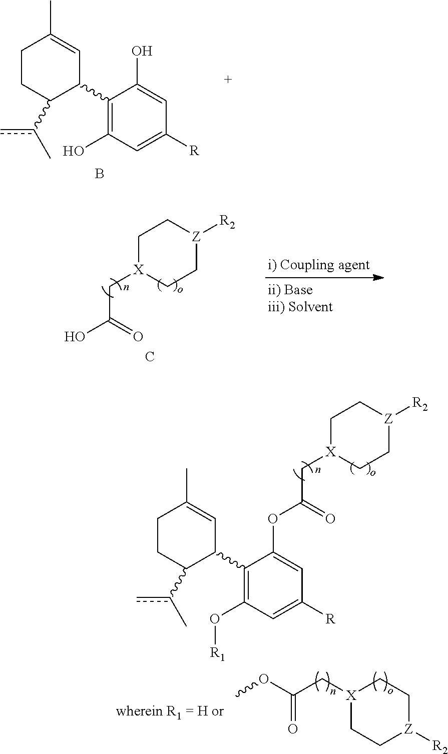

### CROSS REFERENCES TO RELATED APPLICATIONS

This application is a national-stage application under 35 U.S.C. § 371 of International Application No. PCT/IN2023/050656, filed Jul. 6, 2023, which International Application claims benefit of priority to Indian Patent Application number 202211039597, filed Jul. 8, 2022.

### TECHNICAL FIELD OF THE INVENTION

The present invention relates to a prodrug of cannabidiol [CBD]-type phytocannabinoids of formula A, wherein prodrug counterpart is attached through ester bond. Particularly, present invention relates to a process for the preparation of prodrug of formula A and their release studies in ex vivo and in vivo systems, methods of treating the diseases, which are known to cure with cannabidiol.

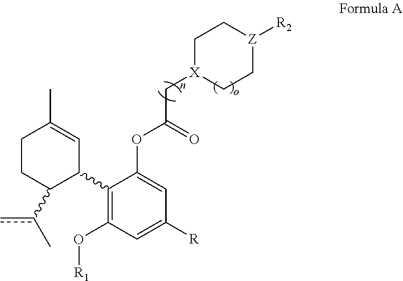

### BACKGROUND OF THE INVENTION

Cannabidiols (CBD) belong to meroterpenoid class, a non-psychotic second most studied phytocannabinoid present in the cannabis. In literature, CBD is reported for the multiple mechanism of actions towards different disease conditions like anti-inflammatory, depression (Nature, 2015, 525, S6) anxiety, insomnia, epilepsy, neurodegenerative diseases (J Pain., 2013, 14, 136), anti-microbial, (Commun. Biol., 2021. 4, 7), pain management in multiple sclerosis and cancer etc., (Cannabinoids, 2016, 11, 1).

Recently, major drug regulatory authorities like USFDA and EMEA authorised the pure CBD based pharmaceutical preparation for the treatment of Dravet syndrome (DS) and Lennox Gastaut syndrome (LGS) with trade name of Epidolex in US and Epidyloex in Europe (Biomed. Pharmacother., 2020, 132, 110889; Clinical Pharmacology and Therapeutics., 2015, 97, 553).

The published pharmacokinetic profile studies of the CBD via oral, subcutaneous, intravenous and pulmonary dosing in different species revealed that CBD has very poor oral bioavailability, very high plasma protein binding (>94%), large volume of distribution (Drugs, 2019, 79, 1435) and high first pass metabolism.

There are >100 phytocannabinoids present in the cannabis, and in the recent decade, CBD-type phytocannabinoids such as cannabidivarin (CBDV) and cannabidiorcinol (CBDO) have also shown interesting pharmacological profile and now also being investigated for the management of several diseases (Epilepsia.2014, 55, 791). Like CBD, other CBD-type phytocannabinoids also have poor drug-likeness because of high lipophilicity.

Taking in view of above facts, in an endeavour to enhancing the oral bioavailability of CBD and CBD-type phytocannabinoids, several attempts were made such as
i) formulation of CBD with appropriate delivery system and
ii) synthesis of their prodrugs. The numbers of patents were elaborated on the prodrugs of CBD and CBD-type phytocannabinoids are discussed below:

J. Med. Chem. 1985, 28, 783 reported the acetylated CBD and its analogs and used the same as an intermediate to modify the terpene part (C-10 position).

WO2008/107879A1, disclosed that, hydroxyl group of resorcinol ring substituted with individually or both with —H, acetyl, ethoxy, 2-yl-acetic acid, ethanol-2-yl, ethanamine-2-yl, N-Boc-ethanamine-2-yl, N-Fmoc-ethanamine-2-yl, 3-morpholinopropanoyl and acetonitrile-2-yl. However, reported examples of CBD having 3-morpholinopropanoyl on one of the hydroxyl groups of resorcinol is having methyl group at another hydroxyl group.

WO2009/018389A1, disclosed the CBD-based prodrugs with polar moieties and having a bio-labile linker such as ester, oxygenated ester, oxa ester, pegylated ester, hydroxylated ester, alkyl ester, amino ester, alkylamino ester, dialkylamino ester, carbonate, alkyl carbonate, carbamate, alkyl carbamate, amino carbamate, alkylamino carbamate, dialkylamino carbamate. Subsequently the application WO2011/026144A1 describes the microneedle formulation of CBD and prodrugs described in the WO2009/018389A1.

US2015/313868A1 disclosed that use of cannabinoid and CBD conjugate with compounds belong to opioids, such asgabapentins, pregablins, benzodiazepines, atropines, oximes, antioxidants, alkalizing agents, terpenes and NSAIDs for the treatment of the symptoms and sign of anticholinesterase toxicity associated with organophosphate (“OP”) and/or carbamate exposure.

WO2017/181118A1 in another attempt stated that, hydroxylated group of aromatic ring substituted with —H, acetyl, propionyl, 3-hydroxy-2-methylpropionyl, TMS, TBDMS, benzyl, —C(O)[CH2]x-C(O)OH, —C(O)[CH2]X—OR, —C(O)[CHR4]X—C(O)OH, —C(O)[CHR4]X—OR5, —C(O)[CR4R5]X—OR6 etc., amino derivatives, a L-amino acid residue, a D-amino acid residue, a γ-amino acid residue, —P(O)[OY](OZ) and —P(O)[NR4NR5](OY).

WO 2018/096504A1 disclosed the CBD-based prodrug with polar moiety having bio-labile linker such as —H, —SO3Na, —PO(ONa)2, —PO(OCH2CH3), —CH2PO(ONa)2, —NO2, -(L)-valine ester, -(L)-N-methyl arginine ester, -α-guanidinoglutaric acid ester, -2-iminobiotin ester, -3-hydroxy anthranilic acid ester, -(L)-nitroarginine ester, -(L)-N5-(1-iminoethyl)-(L)-ornithine (NIO) ester, -(L)-N,N-dimethylarginine ester, -(L)-N6-(1-iminoethyl)-(L)-lysine (NIL) ester, -(L)-N-monomethyl-(L)-arginine (NMMA) ester, —N-amino-(L)-arginine ester, —N-propyl-(L)-arginine ester, —S-methyl-(L)-thiocitrulline ester, -methyl-(L)-NIO ester, -vinyl-(L)-NIO ester, and -propenyl-(L)-NK) ester. Also disclosed the various methods of treating or preventing disease like grave's disease, Hashimoto's thyroiditis and diabetes mellitus.

In the present invention, prodrugs of CBD-type phytocannabinoids, where one or both of the hydroxyl group are attached through ester bond with polar moieties belong to the following but not limited to these such as 2-morpholinylacetic acid, 2-(4-phenylpiperidin-1-yl)acetic acid, 2-(4-hydroxypiperidin-1-yl)acetic acid, 2-(4-benzylpiperidin-1-yl)acetic acid, 2-(4-cyclopropylpiperazin-1-yl)acetic acid, 2-(4-isopropylpiperazin-1-yl)acetic acid, 2-(4-methylpiperazin-1-yl)acetic acid, 2-(4-phenylpiperazin-1-yl)acetic acid, 2-(piperidin-1-yl)acetic acid, 2-(pyrrolidin-1-yl)acetic acid, etc. The present invention is different from the above disclosed prior arts in terms of polar counter parts.

### OBJECTIVE OF THE INVENTION

The main object of the present invention is to provide a prodrugs of CBD-type phytocannabinoids of formula A.

Another object of the present invention is to provides a process to synthesize prodrugs of CBD-type phytocannabinoids of formula A by coupling with polar moieties via ester linkage.

Yet another object of the present invention is to provide prodrugs of CBD-type phytocannabinoids of Formula A to improve plasma exposure and bio-availability of CBD and CBD-type phytocannabinoids.

### BRIEF DESCRIPTION OF THE DRAWINGS

FIG. 1 represents Ex vivo release kinetic study of O-(2-morpholinoacetyl) cannabidiol (A1a).

FIG. 2 represents Ex vivo release kinetic study of O,O-bis-(2-morpholinoacetyl) cannabidiol (A1b).

FIG. 3 represents Ex vivo release kinetic study of O-{2-(4-phenyl piperadin-1-yl)acetyl} cannabidiol (A2a).

FIG. 4 represents Ex vivo release kinetic study of O-{2-(4-hydroxypiperidin-1-yl)acetyl} cannabidiol (A3a).

FIG. 5 represents Ex vivo release kinetic study of O-{2-(4-cyclopropylpiperidin-1-yl)acetyl} cannabidiol (A5a).

FIG. 6 represents Ex vivo release kinetic study of O,O-bis-{2-(4-cyclopropylpiperidin-1-yl)acetyl} cannabidiol (A5b).

FIG. 7 represents Ex vivo release kinetic study of O-{2-(4-isopropylpiperazin-1-yl)acetyl} cannabidiol (A6a).

FIG. 8 represents Ex vivo release kinetic study of O-{2-(4-methylpiperazin-1-yl)acetyl} cannabidiol (A7a).

FIG. 9 represents Ex vivo release kinetic study of O-{2-(4-phenylpiperazin-1-yl)acetyl} cannabidiol (A8a).

FIG. 10 represents Ex vivo release kinetic study of O-{2-(pyrrolidin-1-yl)acetyl} cannabidiol (A10a).

FIG. 11 represents Mean plasma concentration of B1 [CBD] after oral administration of O,O-bis-(2-morpholinoacetyl) cannabidiol (A1b) or cannabidiol (B1) in mice.

### ABBREVIATIONS

1) ACN-Acetonitrile
2) AUC₀₋ₜ—Area under the curve for plasma concentration from zero to last measurable plasma sample time
3) AUC_{0-∞}—Area under the curve for plasma concentration from zero to time infinity
4) CBD—Cannabidiol
5) CD₃OD—Deuterated methanol
6) CDCl₃—Deuterated chloroform
7) CDI—carbonyldiimidazole
8) CH₃OH—Methanol
9) CHCl₃—Chloroform
10) Cl—Clearance after Oral Administration
11) Cₘₐₓ—Maximum Plasma Concentration
12) CYP 450—Cytochrome enzymes
13) DCC—N,N-Dicyclohexylcarbodiimide
14) DCM—Dichloromethane
15) DEPT—Distortionless Enhancement by Polarization Transfer
16) DIC—N,N-Diisopropylcarbodiimide
17) DMAP—Dimethylaminopyridine
18) DMF—Dimethyformamide
19) DMSO—Dimethylsulfoxide
20) DMSO-d⁶—Deuterated dimethylsulfoxide
21) DS—Dravet syndrome
22) EDC—1-Ethyl-3-(3-dimethylaminopropyl)carbodiimide
23) EMEA—European Medical Evaluation Agency
24) Et₃N—triethylamine
25) GW—pharmaceuticals
26) H₂O—Water
27) HPLC—High performance liquid chromatography
28) HRMS—High resolution mass spectrometry
29) IP—Intraperitoneal
30) K₂CO₃—Potassium carbonate
31) MS—Mass spectrometry
32) LGS—Lennox Gastaut syndrome
33) MAH—Marketing authorised holder
34) MeOH—Methanol
35) MHz—Megahertz
36) MRT—Maximum Residence Time
37) nm—Nanometre
38) NMR—Nuclear Magnetic Resonance
39) PDA—Photo diode array
40) PK—Pharmacokinetics
41) PPM—Parts Per Million
42) RBC—Red Blood cells
43) RBF—Round bottom flask
44) R_{f}—Retention factor
45) RP-C18—Reverse phase column 18
46) SRM—Selected reaction monitoring
47) THF—Tetrahydrofuran
48) TLC—Thin Layer Chromatography
49) Tₘₐₓ—Time to Reach Maximum Plasma Concentration
50) T_{1/2}—Elimination half-life
51) TOF—Turnover Frequency
52) UGT—UDP-glucoronosyl-transferases
53) USFDA—United States Food and Drug Administration
54) V_{d}—Volume of Distribution after oral administration

### SUMMARY OF THE INVENTION

Accordingly, present invention provides a prodrug of cannabidiol of Formula A comprising:

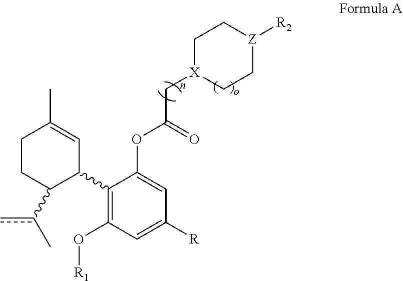

wherein R and R2 are independently selected from the group consisting of H, OH, protected hydroxyl, alkyl, alkenyl, alkynyl, acyl, aryl, heteroaryl, cycloalkyl or heterocycle,
wherein alkyl, alkenyl, alkynyl or acyl with length chain varying from C1 to C14 and is optionally substituted with one or more groups, each independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, NR′R″, S-alkyl, —SO-alkyl, —SO₂-alkyl, S-aryl, —SO-aryl, —SO₂-aryl, —SO₂—N-aryl, —N—SO₂, -arylalkenyl, alkynyl, aryl, heteroaryl, cycloalkyl or heterocycle, wherein the aryl or heteroaryl, whether alone or optionally substituted with one or more substituents independently selected from the group consisting of halogen, OH, alkyl, —O-alkyl, —COOH, —C(O), —C alkyl, —C(O)OC, alkyl or NR′R″;
R′ and R″ are independently selected from the group consisting of H, alkyl, alkenyl, alkynyl or acyl with length chain varying from C1 to C10;
wherein R₁ is selected from

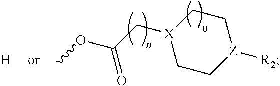

X is selected from C or, N;
Z is selected from C, N or O;
n is independently an integer or a saturated, branched or straight hydrocarbon group having 1 to 10 carbon atoms;
O is independently an integer or a saturated hydrocarbon group having from 0 to 2 carbon atoms when O=0, then it is 5 membered rings, when O=1, then it is 6 membered ring and when O=2, then it is 7 membered rings;
[symbol] represents a single or double bond;
[symbol] represents a single bond which is either above the plane or below the plane.

In an embodiment of the present invention, two stereo-centers are present in the molecule which may be R and S symmetry or mixture thereof.

In another embodiment of the present invention, said prodrug release the parent compound in ex vivo system in the presence of plasma.

In yet another embodiment of the present invention, said prodrug release the parent compound in in vivo mice model after delivering via oral route.

In an embodiment of the present invention, the prodrug compound is selected from the group consisting of;

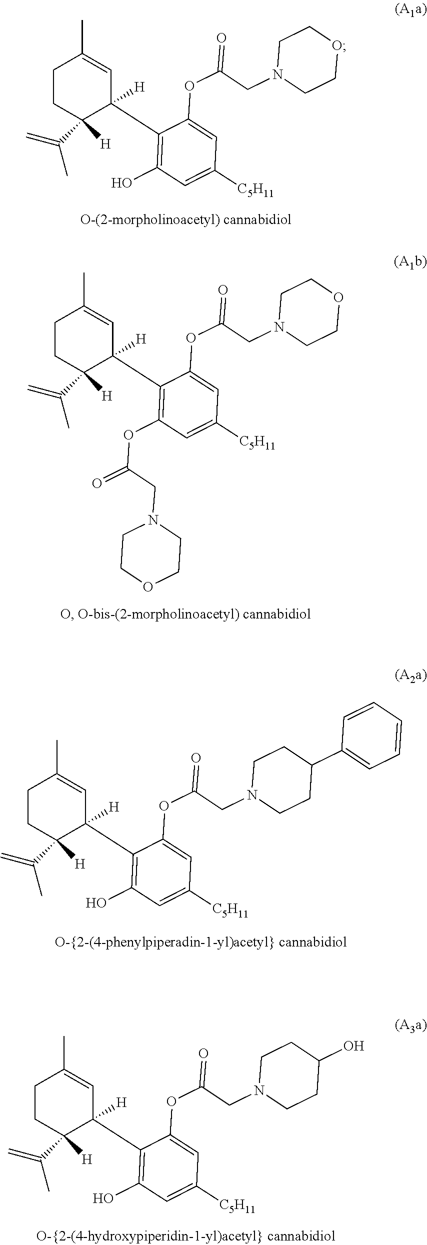

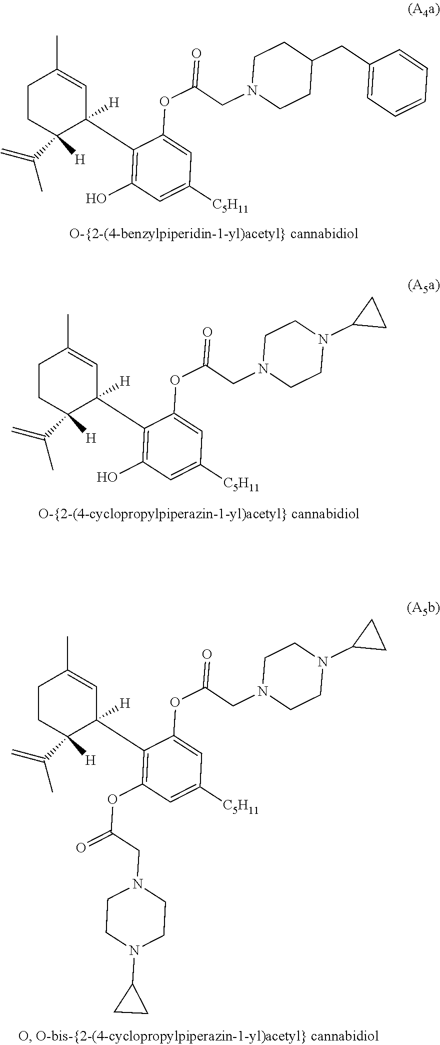

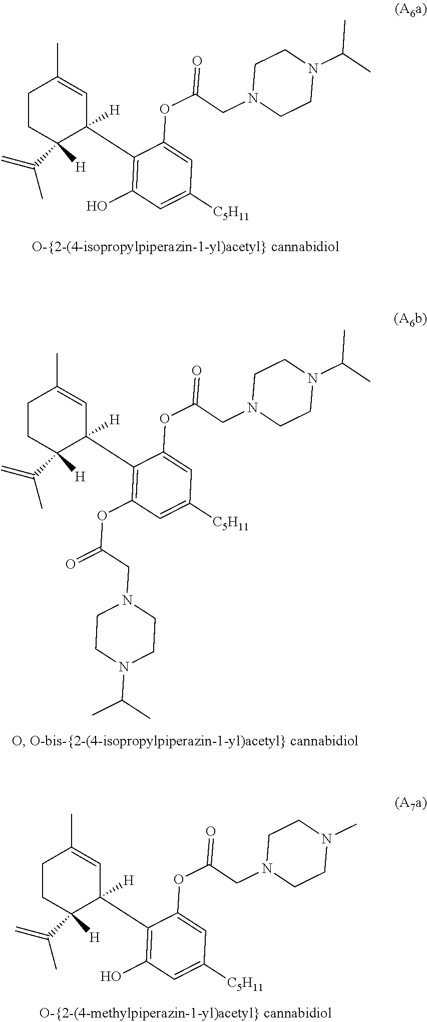

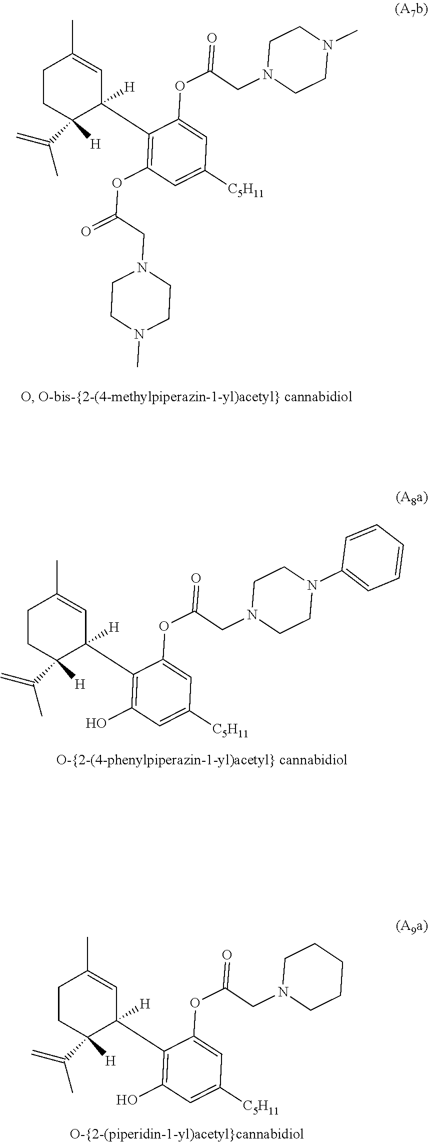

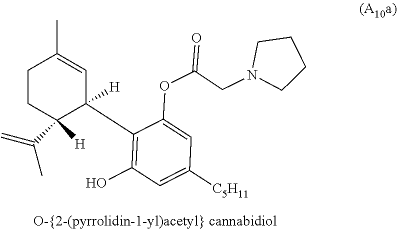

In yet another embodiment, present invention provides a process for preparation of prodrug of Formula A comprising the steps of:
i. reacting fragment B and fragment C in presence of a coupling agent to obtain a reaction mixture;

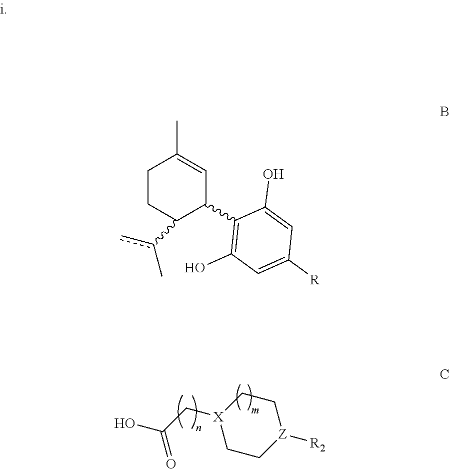

wherein R and R2 are as defined in claim 1;
ii. treating the reaction mixture as obtained in step (i) with base in presence of a dry solvent at temperature in the range of 25-30° C. for a period in the range of 13-16 hours to obtain compound of Formula A.

In yet another embodiment of the present invention, compound of Formula C is selected from the group consisting of:

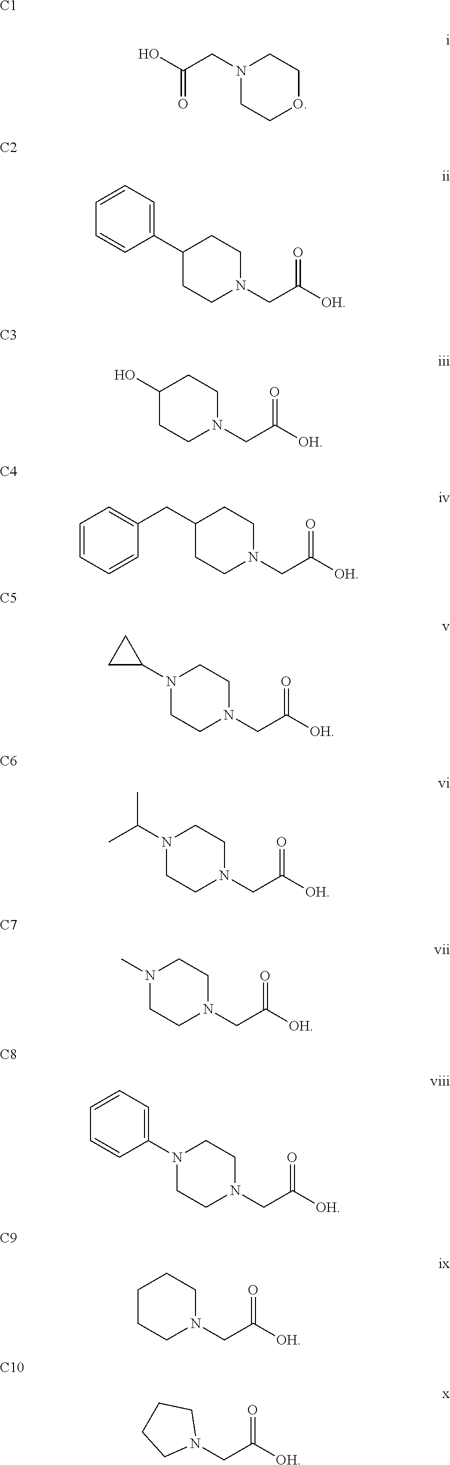

In yet another embodiment of the present invention, the coupling agent is selected from the group consisting of N,N-dicyclohexylcarbodiimide (DCC), N,N-diisopropylcarbodiimide (DIC) and carbonyldiimidazole (CDI) or 1-ethyl-3-(3-dimethylaminopropyl)carbodiimide (EDC).

In yet another embodiment of the present invention, the base is selected from the group consisting of diethylamino pyridine, 2,6-lutidine, triethylamine or diethylamino pyridine. In yet another embodiment of the present invention, the solvent is selected from the group consisting of dichloromethane, chloroform, isopropanol, acetone, acetonitrile either alone or combination thereof.

In other embodiment of the present invention, a pharmaceutical composition comprising the prodrug, wherein the prodrug comprises:

In another embodiment of the present invention, a method for enhancing the bioavailability of prodrug, wherein the method comprises administering the prodrug to the subject.

In yet another embodiment of the present invention, the method is used for the treatment of at least one disease or disorder.

### DETAILED DESCRIPTION OF THE INVENTION

The present invention provides a prodrug of cannabidiol [CBD] of formula A

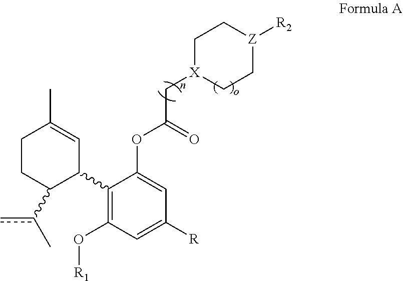

wherein
R and R2 are independently selected from the group consisting of H, OH, protected hydroxyl, alkyl, alkenyl, alkynyl, acyl, aryl, heteroaryl, cycloalkyl or heterocycle,
wherein alkyl, alkenyl, alkynyl or acyl with length chain varying from C1 to C14 and substituted with one or more groups, each independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, NR′R″, S-alkyl, —SO-alkyl, —SO₂-alkyl, S-aryl, —SO-aryl, —SO₂-aryl, —SO₂—N-aryl, —N—SO₂, -arylalkenyl, alkynyl, aryl, heteroaryl, cycloalkyl or heterocycle, wherein the aryl or heteroaryl, whether alone or optionally substituted with one or more substituents independently selected from the group consisting of halogen, OH, alkyl, —O-alkyl, —COOH, —C(O), —C alkyl, —C(O)OC, alkyl or NR′R″; R′ and R″ are independently selected from the group consisting of H, alkyl, alkenyl, alkynyl or acyl with length chain varying from C1 to C10;
wherein R₁ is selected from

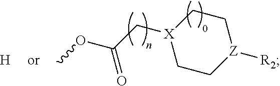

X is selected from C or, N;
Z is selected from C, N or O;
n is independently an integer, or a saturated, branched or straight hydrocarbon group having 1 to 10 carbon atoms;
O is independently an integer, or a saturated hydrocarbon group having from 0 to 2 carbon atoms, when O=0, then it is 5 membered rings, when O=1, then it is 6 membered ring and when O=2, then it is 7 membered ring;
[symbol] represents a single or double bond;

There are two stereo-centre in the molecules, wherein [symbol] represents a single bond it may be above the plane or below the plane.

The stereo-centre may be R an S as well as mixture of both.

The present invention relates to the synthesis of compound of formula A by the coupling of fragment B and fragment C using a coupling agent in presence of a base and dry solvent.

The coupling agent is selected from the group consisting of N,N-dicyclohexylcarbodiimide (DCC), N,N-diisopropylcarbodiimide (DIC) and carbonyldiimidazole (CDI) and most preferably 1-ethyl-3-(3-dimethylaminopropyl)carbodiimide (EDC).

The base is selected from the group consisting of diethylamino pyridine, 2,6-lutidine, triethylamine or diethylamino pyridine either alone or combination thereof.

The solvent is a single solvent or a mixture of one or more solvents and the solvent is selected from the group consisting of dichloromethane, chloroform, isopropanol, acetone, acetonitrile either alone or combination thereof.

The present invention relates to the synthesis of compound of formula A1a namely O-(2-morpholinoacetyl) cannabidiol and A1b namely O,O-bis-(2-morpholinoacetyl) cannabidiol by the coupling of fragment B1 [CS-01] namely cannabidiol and fragment C1 namely 2-morpholinoacetic acid using coupling agent in the presence of base in dry solvent.

The present invention relates to the synthesis of compound of formula A2a namely O-{2-(4-phenylpiperidin-1-yl)acetyl} cannabidiol by the coupling of fragment B1 namely cannabidiol and fragment C2 namely 2-(4-phenylpiperazin-1-yl)acetic acid using coupling agent in the presence of base in dry solvent.

The present invention relates to the synthesis of compound of formula A3a namely O-{2-(4-hydroxypiperidin-1-yl)acetyl} cannabidiol by the coupling of fragment B1 namely cannabidiol and fragment C3 namely 2-(4-hydroxypiperidin-1-yl)acetic acid using coupling agent in the presence of base in dry solvent.

The present invention relates to the synthesis of compound of formula A4a namely O-{2-(4-benzylpiperidin-1-yl)acetyl} cannabidiol by the coupling of fragment B1 namely cannabidiol and fragment C4 namely 2 2-(4-benzylpiperidin-1-yl)acetic acid using coupling agent in the presence of base in dry solvent.

The present invention relates to the synthesis of compound of formula A5a namely O-{2-(4-cyclopropylpiperazin-1-yl)acetyl} cannabidiol and A5b namely O,O-bis-{2-(4-cyclopropylpiperazin-1-yl)acetyl} cannabidiol by the coupling of fragment B1 namely cannabidiol and fragment C5 namely 2-(4-cyclopropylpiperidin-1-yl)acetic acid using coupling agent in the presence of base in dry solvent.

The present invention relates to the synthesis of compound of formula A6a namely O-{2-(4-isopropylpiperazin-1-yl)acetyl} cannabidiol and A6b namely O,O-bis-{2-(4-isopropylpiperazin-1-yl)acetyl} cannabidiol by the coupling of fragment B1 namely cannabidiol and fragment C6 namely 2-(4-isopropylpiperazin-1-yl)acetic acid using coupling agent in the presence of base in dry solvent.

The present invention relates to the synthesis of compound of formula A7a namely O-{2-(4-methylpiperazin-1-yl)acetyl} cannabidiol and A7b namely O,O-bis-{2-(4-methylpiperazin-1-yl)acetyl} cannabidiol by coupling of the fragment B1 namely cannabidiol and fragment C7 namely 2-(4-methylpiperazin-1-yl)acetic acid using coupling agent in the presence of base in dry solvent.

The present invention relates to the synthesis of compound of formula A8a namely O-{2-(4-phenylpiperazin-1-yl)acetyl} cannabidiol by coupling of fragment B1 namely cannabidiol and fragment C8 namely 2-(4-phenylpiperazin-1-yl)acetic acid using coupling agent in the presence of base in dry solvent.

The present invention relates to the synthesis of compound of formula A9a namely O-{2-(piperidin-1-yl)acetyl} cannabidiol by the coupling of fragment B1 namely cannabidiol and fragment C9 namely 2-(piperidin-1-yl)acetic acid using coupling agent in the presence of base in dry solvent.

The present invention relates to the synthesis of compound of formula A10a namely O-{2-(pyrrolidin-1-yl)acetyl} cannabidiol by the coupling of fragment B1 namely cannabidiol and fragment C10 namely 2-(pyrrolidin-1-yl)acetic acid using coupling agent in the presence of base in dry solvent.

The synthesized prodrugs such as O-(2-morpholinoacetyl) cannabidiol (A1a), O,O-bis-(2-morpholinoacetyl) cannabidiol (A1b), O-{2-(4-phenyl piperadin-1-yl)acetyl} cannabidiol (A2a), O-{2-(4-hydroxypiperidin-1-yl)acetyl} cannabidiol (A3a), O-{2-(4-cyclopropylpiperazin-1-yl)acetyl} cannabidiol (A5a), O,O-bis-{2-(4-cyclopropylpiperazin-1-yl)acetyl} cannabidiol (A5b), O-{2-(4-isopropylpiperazin-1-yl)acetyl cannabidiol (A6a), O-{2-(4-isopropylpiperazin-1-yl)acetyl cannabidiol (A6a), O-{2-(4-phenylpiperazin-1-yl)acetyl cannabidiol (A8a) and O-{2-(pyrrolidin-1-yl)acetyl} cannabidiol (A10a) were studied for release kinetic study in ex vivo system using plasma sample, wherein the synthesized prodrugs release the CBD over a period of time.

The synthesized prodrug such as O,O-bis-(2-morpholinoacetyl) cannabidiol (A1b), was studied for release study in in vivo system via oral route using mice animal model. The release of compound CBD (B1) from prodrugs was measured and compared with pure CBD.

All the product mixtures were analyzed by thin layer chromatography.

All prodrugs analyzed by charring regent such as anisaldehyde solution, dragendroff's solution and ninhydrin's solution.

All the reactions were performed under inert atmosphere wherever required.

NMR spectra (¹HNMR, ¹³C, DEPT) were recorded in 400 MHz spectrometer using CDCl₃ and CD₃OD solvent.

ES1-MS and HRMS spectra were recorded on LC-MS/MS and HRMS-6540-UHD machines.

Optical rotations were measured on a Perkin Elmer polarimeter.

Column chromatography was carried out with silica gel (60-120, 100-200 and 230-400 mesh). Ethical approval for the human plasma studies has been taken from IEC, Govt. Medical College, Jammu (Ethical approval number 1EC/GMC/Cat A/2020/267), however, the ethical approval for animal PK study was taken from IAEC IIIM, Jammu (IAEC approval no.-282/80/2022).

### EXAMPLES

The following examples are given by way of illustration only and therefore should not be construed to limit the scope of the present invention in any manner.

### Example 1: Synthesis of CBD-Type Prodrug O-(2-morpholinoacetyl) cannabidiol (A1a) and O,O-bis-(2-morpholinoacetyl) cannabidiol (A1b)

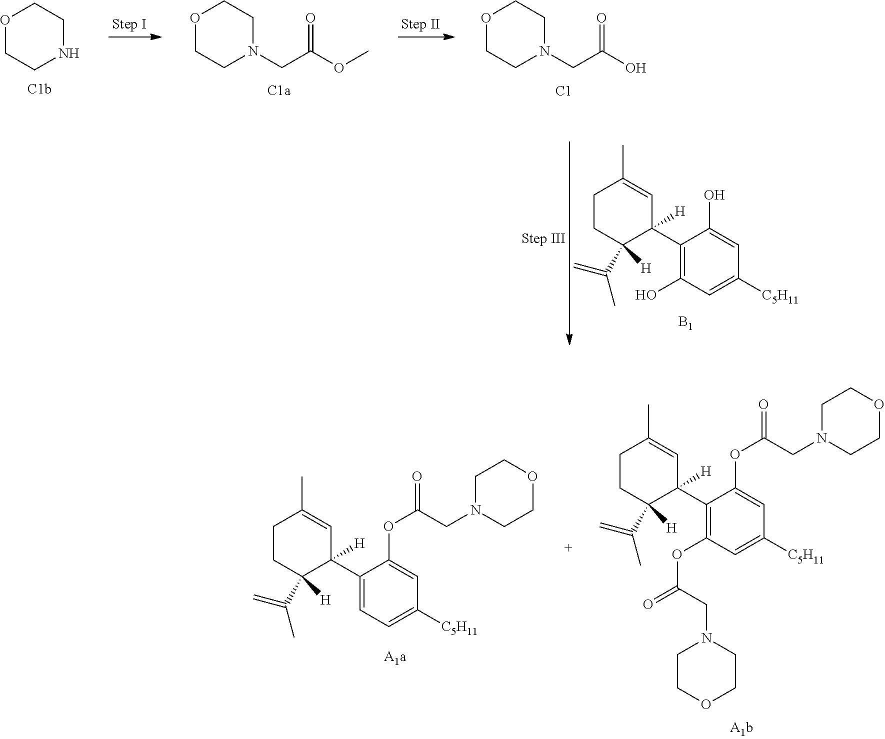

### Step I: Synthesis of methyl 2-morpholinoacetate (C1a)

Morpholine (C1b, 1 g, 1 eq.), methyl 2-bromoacetate (3.496 g, 2 eq.) and triethylamine (TEA) (3.484 g, 3 eq.) (act as base) were taken in round bottom flask (RBF) containing tetrahydrofuran (THF) solvent and stirred for 4 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected, dried over sodium sulphate and evaporated in vacuo. The crude material was purified by using silica gel chromatography eluted in mixture of ethyl acetate:hexane (R_{f}˜0.5/50% Ethyl acetate:Hexane) to get the pure compound (C1a 0.657 g, 35%).

### Step II: Synthesis of 2-morpholinoacetic acid (C1)

Methyl-2-morpholinoacetate (C1a, 0.445 g, 1 eq.) and 2N NaOH (act as base) were taken in RBF containing methanol (MeOH) solvent and stirred for 10 minutes at 25° C. until completion as monitored by TLC. Filtered the reaction mixture and evaporated the solvent through rota-evaporator. Purification was done by using silica gel chromatography eluted in mixture of methanol:chloroform (R_{f}˜0.5/80% methanol:chloroform) to get the pure product (C1, 273 mg, 67%) as white solid compound. The pure compound was characterised by ¹H NMR, MS. ¹H NMR (400 MHz, CDCl₃) δ 6.13 (bs, 1H), 3.77 (s, 4H), 3.19 (s, 2H), 2.78 (s, 4H); MS: (ESI+): m/z calcd for C₆H₁₂NO₃ 146.08; found 146.07.

### Step III: Synthesis of CBD-Type Prodrug O-(2-morpholinoacetyl) cannabidiol (A1a) and O,O-bis-(2-morpholinoacetyl) cannabidiol (A1b)

To a stirred solution of cannabidiol (B1, 200 mg, 0.636 mmol) and 2-morpholinoacetic acid (C1, 184 mg, 1.272 mmol) in DCM/THF (1:1) and added DMAP (61 mg, 0.508 mmol), followed by 1-ethyl-3-(3-dimethylaminopropyl)carbodiimide (EDC) (364 mg, 1.908 mmol) under nitrogen atmosphere and stirred for 15 hours at 25° C. Reaction was monitored by TLC until reactant consumed. Two products (Mono and di morpholine CBD derivatives) were formed in the reaction at different Rr values i.e., R_{f}˜0.7/20% ethyl acetate:hexane and R_{f}˜0.3/20% ethyl acetate:Hexane respectively. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. These compounds were separated/purified by using silica gel chromatography eluted in mixture of ethyl acetate:hexane to get the mono-morpholine CBD product i.e., O-(2-morpholinoacetyl) cannabidiol (A1a, 90 mg, 32%) and di-morpholine CBD product O,O-(2-morpholinoacetyl) cannabidiol (A1b, 147 mg, 40%).

These derivatives were characterized by ¹H NMR, ¹³C NMR, DEPT and HRMS. ¹H NMR (400 MHz, CDCl₃) δ 6.55 (s, 1H), 6.42 (s, 1H), 5.98 (br s, OH), 5.52 (s, 1H), 4.60 (s, 1H), 4.45 (s, 1H), 3.77 (t, J=4.7 Hz, 4H), 3.50-3.45 (m, 1H), 3.42-3.33 (m, 2H), 2.70-2.62 (m, 4H), 2.50-2.43 (m, 3H), 2.22-2.04 (m, 2H), 1.86-1.78 (m, 2H), 1.77 (s, 3H), 1.60 (s, 3H), 1.59-1.54 (m, 2H), 1.33-1.26 (m, 4H), 0.88 (t, J=6.9 Hz, 3H); ¹³C {¹H} NMR (101 MHz, CDCl₃) δ 70.81, 157.96, 151.41, 150.02, 143.29, 133.54, 127.18, 122.15, 115.46, 114.35, 111.29, 67.96, 60.46, 54.73, 47.54, 38.43, 36.60, 32.78, 32.03, 30.84, 23.95, 23.75, 23.50, 19.82, 14.61; [a]_{D}²⁰=−72 (c=1.0, MeOH); HRMS: (M+H)⁺: m/z calcd for C₂₇H₄₀NO₄ 442.2957; found 442.2957. ¹H NMR (400 MHz, CDCl₃) δ 6.76 (s, 2H), 5.22 (s, 1H), 4.56 (s, 1H), 4.48 (s, 1H), 3.80-3.78 (m, 8H), 3.53-3.50 (m, 1H), 3.46-3.35 (m, 4H), 2.75-2.63 (m, 8H), 2.62-2.51 (m, 3H), 2.16-2.03 (m, 2H), 1.79-1.72 (m, 2H), 1.68 (s, 3H), 1.65-1.59 (m, 2H), 1.58 (s, 3H), 1.34-1.25 (m, 4H), 0.90 (t, J=6.9 Hz, 3H); ¹³C {¹H} NMR (101 MHz, CDCl₃) δ 170.28, 151.04, 149.26, 143.83, 134.45, 127.80, 125.76, 122.19, 112.00, 67.92, 60.25, 54.65, 47.31, 40.10, 36.31, 32.68, 31.90, 30.41, 24.19, 23.77, 20.36, 14.67; [a]_{D}²⁰=−75 (c=1.0, MeOH); HRMS: (M+H)⁺: m/z calcd for C₃₃H₄₉N₂O₆ 569.3591; found 569.3591.

### Example 2: Synthesis of CBD-Type Prodrug O-{2-(4-phenylpiperidin-1-yl)acetyl} cannabidiol (A2a)

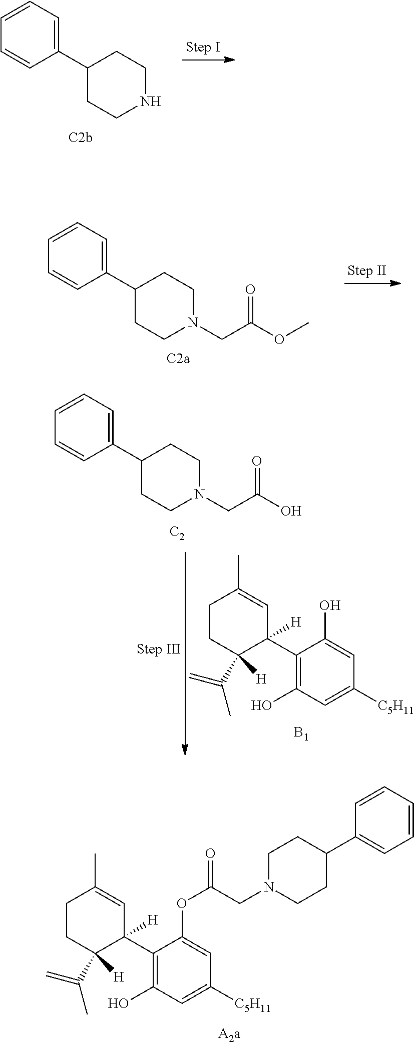

### Step 1: Synthesis of methyl 2-(4-phenylpiperidin-1-yl)acetate (C2a)

Phenyl piperadine (C2b, 0.6 g, 1 eq.), methyl 2-bromoacetate (1.132 g, 2 eq.) and triethylamine (TEA) (1.881 g, 3 eq.) (act as base) were taken in RBF containing THE solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 times with ethyl acetate. Organic layers were collected, dried over sodium sulphate and evaporated in vacuo. The crude material was purified by using silica gel chromatography eluting in mixture of ethyl acetate:hexane (R_{f}˜0.5/50% ethyl acetate:hexane) to get the pure compound (C2a, 0.7 g, 45%).

Step 2: Synthesis of 2-(4-phenylpiperidin-1-yl)acetic acid (C2)

Methyl 2-(4-phenylpiperidin-1-yl)acetate (C2a, 0.7 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography eluted in mixture of methanol:chloroform (R_{f}˜0.5/95% methanol:chloroform) to get the pure product (C2, 0.5 g, 80%) as white solid compound. The pure compound was characterised by ¹H NMR, MS.

¹H NMR (400 MHz, CDCl₃) δ 7.33-7.11 (m, 5H), 3.59-3.56 (m, 2H), 3.49 (s, 1H), 2.99-2.77 (m, 4H), 2.02-1.97 (m, 4H); MS: (ESI+): m/z calcd for C₁₃H₁₈NO₂ 220.13; found 220.25.

### Step 3: Synthesis of CBD-Type Prodrug O-{2-(4-phenyl piperadin-1-yl)acetyl} cannabidiol (A2a)

To a stirred solution of cannabidiol (B1, 200 mg, 0.636 mmol) and 2-(4-phenylpiperidin-1-yl)acetic acid (C2, 276 mg, 1.272 mmol) in DCM/THF (1:1) and added DMAP (61 mg, 0.508 mmol), followed by 1-ethyl-3-(3-dimethylaminopropyl)carbodiimide (EDC) (364 mg, 1.908 mmol) under nitrogen atmosphere and stirred for 14 hours at 28° C. Reaction was monitored by TLC until reactant consumed. Reaction mixture was dried in vacuo. The concentrated reaction mixture was workup with ethyl acetate and sodium bicarbonate solution. Aqueous layer was washed 2 or 3 times with ethyl acetate. Organic layer was compiled, dried over sodium sulphate and evaporated in vacuo. This compound was separated/purified by using silica gel chromatography in mixture of ethyl acetate:hexane (R_{f}˜0.5/20% ethyl acetate:hexane) to get the mono-phenylpiperidine CBD product i.e., O-{2-(4-phenyl piperadin-1-yl)acetyl} cannabidiol (A2, 80 mg, 24%) as light yellow oil.

This derivative was characterized by ¹H NMR, ¹³C NMR, DEPT and HRMS. ¹H NMR (400 MHz, CDCl₃) δ 7.33-7.16 (m, 5H), 6.55 (s, 1H), 6.44 (s, 1H), 5.53 (s, 1H), 4.61 (s, 1H), 4.46 (s, 1H), 3.59-3.50 (m, 1H), 3.50-3.36 (m, 2H), 3.17-3.09 (m, 2H), 2.57-2.36 (m, 6H), 2.21-2.03 (m, 2H), 1.96-1.81 (m, 6H), 1.76 (s, 3H), 1.62 (s, 3H), 1.59-1.53 (m, 2H), 1.28 (m, 4H), 0.88 (t, J=6.7 Hz, 3H); ¹³C {¹H} NMR (101 MHz, CDCl₃) δ 170.91, 157.97, 151.41, 149.96, 147.56, 143.24, 129.76, 128.14, 127.61, 122.08, 115.59, 113.99, 111.41, 60.54, 55.60, 46.61, 43.50, 37.77, 36.64, 34.39, 32.96, 32.09, 30.93, 30.77, 30.44, 24.28, 23.98, 19.80, 14.72; [a]_{D}²⁰=−65 (c=1.0, MeOH); HRMS: (M+H)⁺: m/z calcd for C₃₄H₄₆NO₃ 516.3478; found 516.3478.

### Example 3: Synthesis of CBD-Type Prodrug O-{2-(4-hydroxypiperidin-1-yl)acetyl} cannabidiol (A3a)

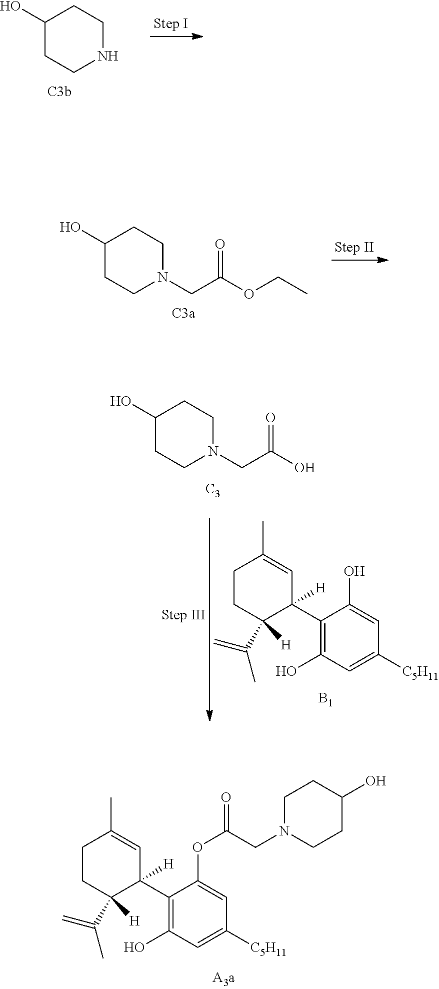

### Step 1: Synthesis of ethyl 2-(4-hydroxypiperidin-1-yl)acetate (C3a)

Piperidin-4-ol (C3b, 1.5 g, 1 eq.) and ethyl 2-chloroacetate (3.623 g, 2 eq.) and potassium carbonate (K₂CO₃) (6.148 g, 3 eq.) (act as base) were taken in RBF containing ACN solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layers were collected, dried over sodium sulphate and evaporated in vacuo. The crude material was purified by using silica gel chromatography in mixture of ethyl acetate:hexane (R_{f}˜0.5/50% ethyl acetate:hexane) to get the pure compound (C3a, 2.5 g, 90%).

### Step 2: Synthesis of 2-(4-hydroxypiperidin-1-yl)acetic acid (C3)

Ethyl 2-(4-hydroxypiperidin-1-yl)acetate (C3a, 2.230 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography in mixture of methanol:chloroform (R_{f}˜0.5/95% methanol:chloroform) to get the pure product (C3, 1.700 g, 89%) as white solid compound. The pure compound was characterised by ¹H NMR, MS. ¹H NMR (400 MHz, MeOD) δ 3.92-3.83 (m, 1H), 3.82-3.72 (m, 1H), 3.36 (s, 2H), 3.23-3.21 (m, 2H), 2.92 (s, 2H), 2.03-1.86 (m, 2H), 1.78-1.62 (m, 2H); MS: (ESI+): m/z calcd for C₇H₁₄NO₃ 160.09; found 160.50.

### Step 3: Synthesis of CBD-Type Prodrug O-{2-(4-hydroxypiperidin-1-yl)acetyl} cannabidiol (A3a)

To a stirred solution of cannabidiol (B1, 300 mg, 0.955 mmol) and 2 2-(4-hydroxypiperidin-1-yl)acetic acid (C3, 334 mg, 2.101 mmol) in DCM/THF (1:1) and added DMAP (91 mg, 0.764 mmol), followed by 1-ethyl-3-(3-dimethylaminopropyl)carbodiimide (EDC) (455 mg, 2.387 mmol) under nitrogen atmosphere and stirred for 13 hours at 28° C. Reaction was monitored by TLC until reactant consumed. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. This compound was separated/purified by using silica gel chromatography in mixture of ethyl acetate:hexane (R_{f}˜0.5/70% ethyl acetate:hexane) to get the mono-hydroxypiperidine CBD product i.e., O-{2-(4-hydroxypiperidin-1-yl)acetyl} cannabidiol (A3a, 80 mg, 18%) as light yellow oil.

This derivative was characterized by ¹H NMR, ¹³C NMR, DEPT and HRMS. ¹H NMR (400 MHz, CDCl₃) δ 6.53 (s, 1H), 6.40 (s, 1H), 6.02 (br s, OH), 5.50 (s, 1H), 4.58 (s, 1H), 4.44 (s, 1H), 3.73-3.72 (m, 1H), 3.65-3.49 (m, 1H), 3.45-3.41 (m, 2H), 2.89-2.86 (m, 2H), 2.58-2.49 (m, 2H), 2.47-2.45 (m, 3H), 2.19-2.02 (m, 2H), 1.94-1.78 (m, 4H), 1.75 (s, 3H), 1.67-1.64 (m, 2H), 1.59 (s, 3H), 1.56 (s, 2H), 1.28 (s, 4H), 0.87 (t, J=5.6 Hz, 3H); ¹³C {¹H} NMR (101 MHz, CDCl₃) δ 170.93, 157.95, 151.42, 149.99, 143.26, 133.46, 127.18, 122.13, 115.47, 114.13, 111.31, 68.04, 60.20, 52.71, 51.84, 47.58, 38.25, 36.59, 34.97, 32.79, 32.03, 30.84, 24.15, 23.80, 19.78, 14.64; [a]_{D}²⁰=−41 (c=1.0, MeOH); HRMS: (M+H)⁺: m/z calcd for C₂₈H₄₂NO₄ 456.3114; found 456.3114.

### Example 4: Synthesis of CBD-Type Prodrug O-{2-(4-benzylpiperidin-1-yl)acetyl} cannabidiol (A4)

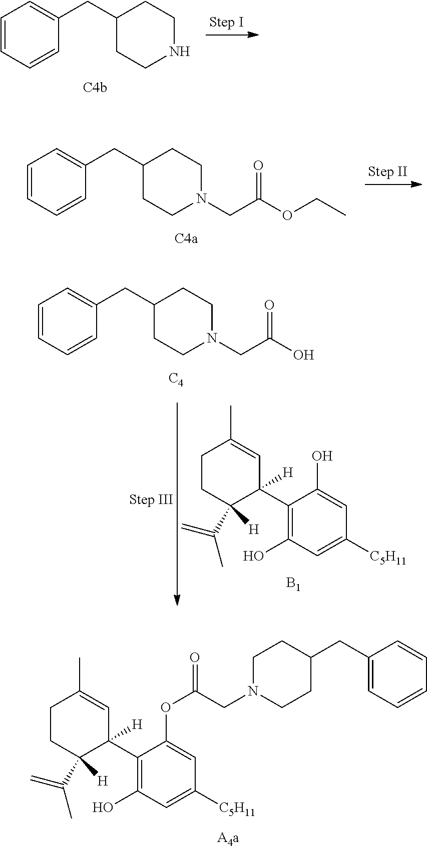

### Step 1: Synthesis of ethyl 2-(4-benzylpiperidin-1-yl)acetate (C4a)

4-Benzylpiperidine (C4b, 1.5 g, 1 eq.) and ethyl 2-chloroacetate (2.091 g, 2 eq.) and potassium carbonate (K₂CO₃) (3.548 g, 3 eq.) (act as base) were taken in RBF containing ACN solvent and stirred for 4 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected, dried over sodium sulphate and evaporated in vacuo. The crude material was purified by using silica gel chromatography eluted in mixture of ethyl acetate:hexane (R_{f}˜0.5/50% ethyl acetate:hexane) to get the pure compound (C4a, 1.640 g, 73%).

### Step 2: Synthesis of 2-(4-benzylpiperidin-1-yl)acetic acid (C4)

Ethyl 2-(4-benzylpiperidin-1-yl)acetate (C4a, 1.640 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography eluted in mixture of methanol:chloroform (R_{f}˜0.5/60% methanol:chloroform) to get the pure product (C4, 1.253 g, 85%) as white solid compound. The pure compound was characterised by ¹H NMR, MS.

¹H NMR (400 MHz, MeOD) δ 7.25-6.99 (m, 5H), 3.49-6.45 (m, 4H), 2.86-2.80 (m, 2H), 2.51-2.50 (m, 2H), 1.75-1.72 (m, 3H), 1.53-1.44 (m, 2H); MS: (ESI+): m/z calcd for C₁₄H₂₀NO₂ 234.14; found 234.50.

### Step 3: Synthesis of CBD-Type Prodrug O-{2-(4-benzylpiperidin-1-yl)acetyl} cannabidiol (A4a)

To a stirred solution of cannabidiol (B1, 300 mg, 0.955 mmol) and 2-(4-benzylpiperidin-1-yl)acetic acid (C4, 489 mg, 2.101 mmol) in DCM/THF (1:1) and added DMAP (91 mg, 0.764 mmol), followed by 1-ethyl-3-(3-dimethylaminopropyl)carbodiimide (EDC) (455 mg, 2.387 mmol) under nitrogen atmosphere and stirred for 16 hours at 28° C. Reaction was monitored by TLC until reactant consumed. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. This compound was separated/purified by using silica gel chromatography eluted in mixture of ethyl acetate:hexane (R_{f}˜0.5/40% ethyl acetate:hexane) to get the mono-benzylpiperidine CBD product i.e., O-{2-(4-benzylpiperidin-1-yl)acetyl} cannabidiol (A4a, 30 mg, 6%) as light yellow oil.

This derivative was characterized by ¹H NMR, ¹³C NMR, DEPT and HRMS. ¹H NMR (400 MHz, CDCl₃) δ 7.29-7.13 (m, 5H), 6.54 (s, 1H), 6.41 (s, 1H), 5.99 (br s, OH), 5.51 (s, 1H), 4.59 (s, 1H), 4.44 (s, 1H), 3.51 (s, 1H), 3.42-3.32 (m, 2H), 3.04-2.94 (m, 2H), 2.58-2.53 (m, 2H), 2.51-2.44 (m, 3H), 2.37-2.16 (m, 4H), 2.15-2.02 (m, 2H), 1.78 (d, J=12.2 Hz, 5H), 1.66 (d, J=12.1 Hz, 3H), 1.60 (s, 3H), 1.45-1.38 (m, 2H), 1.30 (s, 4H), 0.87 (t, J=10.8 Hz, 3H; ¹³C {¹H} NMR (101 MHz, CDCl₃) δ 170.79, 157.95, 149.99, 143.26, 141.89, 133.51, 130.41, 129.52, 127.20, 122.12, 115.45, 114.20, 111.26, 60.49, 55.13, 44.17, 38.83, 38.39, 36.59, 32.94, 32.91, 32.02, 30.84, 24.15, 23.79, 19.79, 14.63; [a]_{D}²⁰=−56 (c=1.0, MeOH); HRMS: (M+H)⁺: m/z calcd for C₃₅H₄₈NO₃ 530.3634; found 530.3634.

### Example 5: Synthesis of CBD-Type Prodrug O-{2-(4-cyclopropylpiperazin-1-yl)acetyl} cannabidiol (A5a) and O,O-bis-{2-(4-cyclopropylpiperazin-1-yl)acetyl} cannabidiol (A5b)

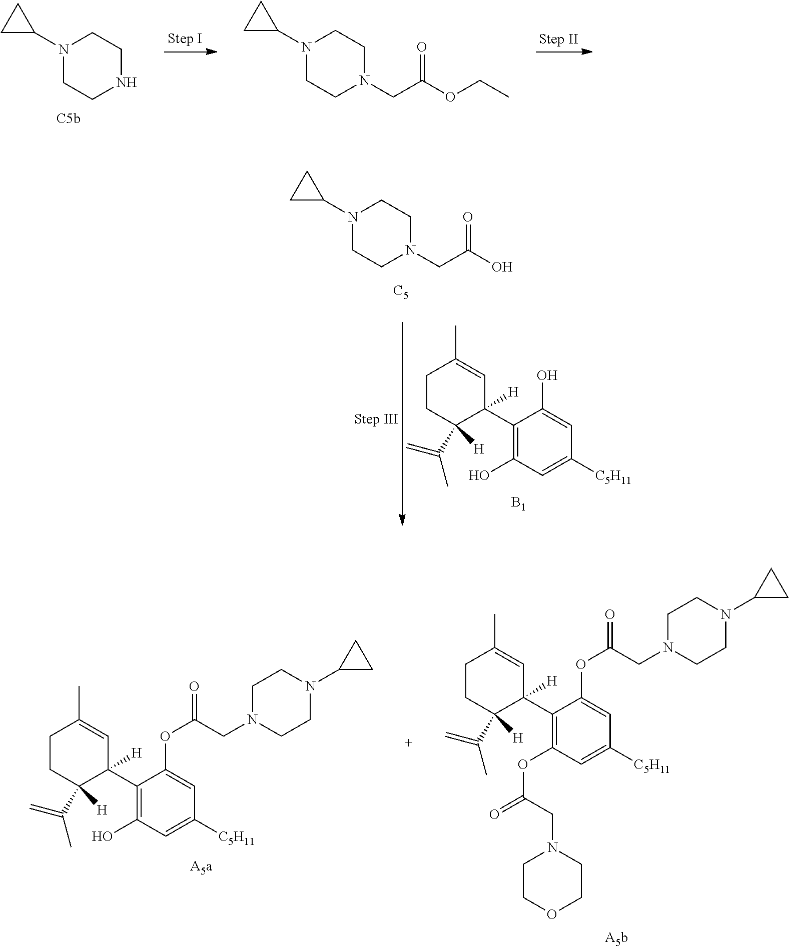

### Step 1: Synthesis of ethyl 2-(4-cyclopropylpiperazin-1-yl)acetate (C5a)

1-Cyclopropylpiperazine (C5b, 1 g, 1 eq.) and ethyl 2-chloroacetate (1.936 g, 2 eq.) and potassium carbonate (K₂CO₃) (3.285 g, 3 eq.) (act as base) were taken in RBF containing ACN solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected, dried over sodium sulphate and evaporated in vacuo. The crude material was purified by using silica gel chromatography eluted in mixture of methanol:chloroform (R_{f}˜0.5/10% methanol:chloroform) to get the pure compound (C5a, 1.407 g, 83%)

### Step 2: Synthesis of 2-(4-cyclopropylpiperazin-1-yl)acetic acid (C5)

Ethyl 2-(4-cyclopropylpiperidin-1-yl)acetate (C5a, 1.407 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography eluted in mixture of methanol:chloroform (TLC R_{f}˜0.5/50% methanol:chloroform) to get the pure product (C5, 1.200 g, 98%) as dark brown semi-solid compound.

The pure compound was characterised by ¹H NMR, MS. ¹H NMR (400 MHz, CDCl₃) δ 5.09 (s, 4H), 3.22 (s, 2H), 3.08 (s, 4H), 2.02 (s, 1H), 0.78-0.69 (m, 4H); MS: (ESI+): m/z calcd for C₉H₁₇N₂O₂ 185.12; found 185.15.

### Step 3: Synthesis of CBD-Type Prodrug O-{2-(4-cyclopropylpiperazin-1-yl)acetyl} cannabidiol (A5a) and O,O-{2-(4-cyclopropylpiperazin-1-yl)acetyl} cannabidiol (A5b)

To a stirred solution of cannabidiol (B1, 200 mg, 0.636 mmol) and 2-(4-cyclopropylpiperazin-1-yl)acetic acid (C5, 234 mg, 1.272 mmol) in DCM/THF (1:1) and added DMAP (61 mg, 0.508 mmol), followed by 11-ethyl-3-(3-dimethylaminopropyl)carbodiimide (EDC) (485 mg, 2.544 mmol) under nitrogen atmosphere and stirred for 14 hours at 25° C. Reaction was monitored by TLC until reactant consumed. Two products (Mono and di-cyclopropylpiperazine CBD derivatives) were formed in the reaction at different Rr values i.e., R_{f}˜0.7/20% ethyl acetate:hexane and R_{f}˜0.3/20% ethyl acetate:hexane respectively. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. These compounds were separated/purified by using silica gel chromatography eluted in mixture of ethyl acetate:hexane to get the mono-cyclopropylpiperazine CBD product i.e., O-{2-(4-cyclopropylpiperidin-1-yl)acetyl} cannabidiol (A5a, 60 mg, 19%) and di-cyclopropylpiperazine CBD product i.e., O,O-{2-(4-cyclopropylpiperidin-1-yl)acetyl} cannabidiol (A5b, 222 mg, 54%) as light yellow oil respectively.

These derivatives were characterized by ¹HNMR, ¹³C NMR, DEPT and HRMS. ¹H NMR (400 MHz, CDCl₃) δ 6.50 (s, 1H), 6.37 (s, 1H), 5.47 (s, 1H), 4.55 (s, 1H), 4.40 (s, 1H), 3.38 (m, 3H), 2.71-2.57 (m, 8H), 2.43 (m, 3H), 2.16-2.02 (m, 2H), 1.94 (d, J=16.8 Hz, 1H), 1.72 (s, 3H), 1.63-1.59 (m, 2H), 1.58-1.51 (m, 5H), 1.23 (m, 4H), 0.83 (t, J=6.6 Hz, 3H), 0.42-0.37 (m, 4H); ¹³C {¹H} NMR (101 MHz, CDCl₃) δ 204.33, 170.77, 157.95, 151.41, 149.98, 143.23, 133.47, 127.46, 122.12, 115.54, 114.16, 111.33, 60.13, 54.16, 53.89, 47.61, 39.69, 38.36, 36.61, 32.80, 32.04, 30.83, 24.17, 23.80, 19.82, 14.65, 6.09; [a]_{D}²⁰=−74 (c=1.0, MeOH); HRMS: (M+H)⁺: m/z calcd for C₃₀H₄₅N₂O₃ 481.3430; found 481.3430. ¹H NMR (400 MHz, CDCl₃) δ 6.73 (s, 2H), 5.19 (s, 1H), 4.53 (s, 1H), 4.45 (s, 1H), 3.51-3.48 (m, 1H), 3.43-3.39 (m, 2H), 3.30-3.16 (m, 2H), 2.71-2.59 (m, 16H), 2.54-2.44 (m, 3H), 2.16-1.97 (m, 2H), 1.82-1.71 (m, 2H), 1.69-1.67 (m, 2H), 1.65 (s, 3H), 1.59-1.57 (m, 2H), 1.55 (s, 3H), 1.32-1.25 (m, 4H), 0.88 (t, J=6.7 Hz, 3H), 0.47-0.44 (m, 4H), 0.41-0.38 (m, 4H); ¹³C {¹H} NMR (101 MHz, CDCl₃) δ 170.36, 151.10, 149.37, 143.87, 134.46, 127.80, 125.80, 122.13, 115.42, 114.28, 111.95, 59.87, 54.21, 53.55, 47.33, 40.19, 39.76, 36.32, 32.66, 31.90, 30.46, 24.15, 23.75, 20.40, 14.61, 5.98; [a]_{D}²⁰=−74 (c=1.0, MeOH); HRMS: (M+H)⁺: m/z calcd for C₃₉H₅₉N₄O₄ 647.4536; found 647.4536.

### Example 6: Synthesis of CBD-Type Prodrug O-{2-(4-isopropylpiperazin-1-yl)acetyl} cannabidiol (A6a) and O,O-bis-{2-(4-isopropylpiperazin-1-yl)acetyl} cannabidiol (A6b)

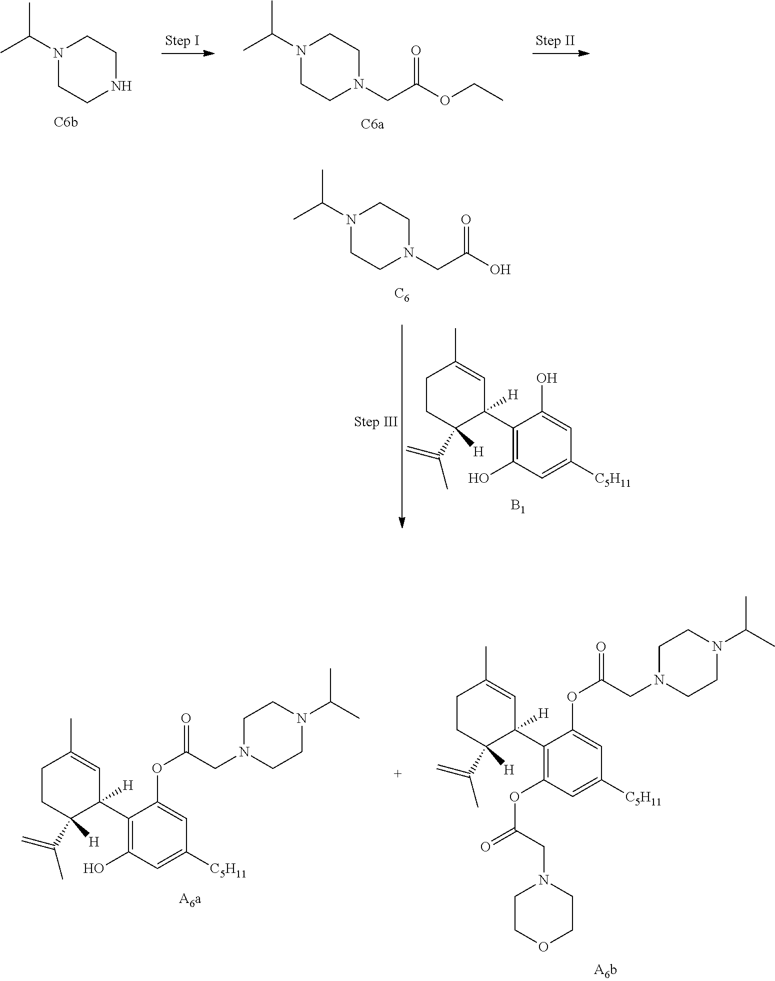

### Step 1: Synthesis of ethyl 2-(4-isopropylpiperazin-1-yl)acetate (C6a)

1-iso-Propylpiperazine (C6b, 1 g, 1 eq.) and ethyl 2-chloroacetate (1.906 g, 2 eq.) and potassium carbonate (K₂CO₃) (3.234 g, 3 eq.) (act as base) were taken in RBF containing ACN solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected, dried over sodium sulphate and evaporated in vacuo. The crude material was purified by using silica gel chromatography eluted in mixture of methanol:chloroform (R_{f}˜0.5:10% methanol:chloroform) to get the pure compound (C6a, 1.339 g, 80%).

### Step 2: Synthesis of 2-(4-iso-propylpiperazin-1-yl)acetic acid (C6)

Ethyl 2-(4-iso-propylpiperazin-1-yl)acetate (C6a, 1.290 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography eluted in mixture of methanol:chloroform (R_{f}˜0.5/95% methanol:chloroform) to get the pure product (C6, 0.989 g, 88%) as white solid compound. The pure compound was characterised by ¹H NMR, MS. ¹H NMR (400 MHz, CDCl₃) δ 4.19 (s, 4H), 2.49 (s, 1H), 2.16 (s, 4H), 0.66 (s, 2H), 0.50 (d, J=6.4 Hz, 6H); MS: (ESI+): m/z calcd for C₉H₁₉N₂O₂ 187.14; found 187.35.

### Step 3: Synthesis of CBD-Type Prodrug O-{2-(4-iso-propylpiperazin-1-yl)acetyl cannabidiol (A6a) and O,O-bis-{2-(4-iso-propylpiperazin-1-yl)acetyl cannabidiol (A6b)

To a stirred solution of cannabidiol (B1, 200 mg, 0.636 mmol) and 2-(4-iso-propylpiperazin-1-yl)acetic acid (C6, 236 mg, 1.272 mmol) in DCM/THF (1:1) and added DMAP (61 mg, 0.508 mmol, followed by 1-ethyl-3-(3-dimethylaminopropyl)carbodiimide (EDC) (485 mg, 2.544 mmol) under nitrogen atmosphere and stirred for 16 hours at 26° C. Reaction was monitored by TLC until reactant consumed. Two products (Mono and di-isopropylpiperazine CBD derivatives) were formed in the reaction at different Rr values i.e., R_{f}˜0.7/10% ethyl acetate:hexane and R_{f}˜0.3/10% ethyl acetate:hexane respectively. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. These compounds were separated/purified by using silica gel chromatography eluted in mixture ethyl acetate:hexane to get the mono-cyclopropyl piperazine CBD product i.e., O-{2-(4-iso-propylpiperazin-1-yl)acetyl cannabidiol (A6a, 70 mg, 22%) and di-cyclopropylpiperazine CBD product i.e., O,O-{2-(4-iso-propylpiperazin-1-yl)acetyl cannabidiol (A6b, 130 mg, 31%) as light yellow oil respectively.

These derivatives were characterized by ¹H NMR, ¹³C NMR, DEPT and HRMS. ¹H NMR (400 MHz, CDCl₃) δ 6.53 (s, 1H), 6.41 (s, 1H), 5.51 (s, 1H), 4.58 (s, 1H), 4.44 (s, 1H), 3.65-3.45 (m, 1H), 3.43-3.28 (m, 2H), 2.79-2.64 (m, 8H), 2.52-2.43 (m, 3H), 2.33-2.07 (m, 2H), 2.05-2.01 (m, 1H), 1.86-1.68 (m, 5H), 1.61 (s, 3H), 1.58-1.52 (m, 2H), 1.31-1.26 (m, 4H), 1.08 (s, 3H), 1.07 (s, 3H), 0.87 (t, J=6.9 Hz, 3H); ¹³C {¹H} NMR (101 MHz, CDCl₃) δ 170.75, 157.94, 151.39, 149.90, 143.23, 133.34, 127.34, 122.10, 115.53, 114.08, 111.35, 60.02, 56.36, 54.02, 49.73, 47.34, 38.21, 36.62, 32.81, 32.11, 32.00, 30.83, 24.21, 23.83, 19.73, 18.89, 14.67; [a]_{D}²⁰=−36 (c=1.0, MeOH); HRMS: (M+H)⁺: m/z calcd for C₃₀H₄₇N₂O₃484.3587; found 484.3587. ¹H NMR (400 MHz, CDCl₃) δ 6.75 (s, 2H), 5.20 (s, 1H), 4.54 (s, 1H), 4.46 (s, 1H), 3.53-3.46 (m, 1H), 3.42-3.36 (m, 2H), 3.34-3.24 (m, 2H), 2.85-2.69 (m, 16H), 2.63-2.51 (m, 3H), 2.31-2.08 (m, 2H), 2.06-1.94 (m, 2H), 1.83-1.72 (m, 2H), 1.67 (s, 3H), 1.62-1.60 (m, 2H), 1.57 (s, 3H), 1.32-1.27 (m, 4H), 1.13 (s, 6H), 1.11 (s, 6H), 0.89 (t, J=6.6 Hz, 3H); ¹³C {¹H} NMR (101 MHz, CDCl₃) δ 170.35, 151.06, 149.34, 143.89, 134.48, 127.81, 125.73, 122.21, 112.00, 59.72, 56.73, 53.60, 49.73, 47.37, 40.09, 36.33, 32.69, 31.92, 30.47, 24.21, 23.80, 20.32, 18.71, 14.71; [a]_{D}²⁰=−42 (c=1.0, MeOH); HRMS: (M+H)⁺: m/z calcd for C₃₉H₆₃N₄O₄ 651.4849; found 651.4849.

### Example 7: Synthesis of CBD-Type Prodrug O-{2-(4-methylpiperazin-1-yl)acetyl cannabidiol (A7a) and O,O-bis {2-(4-methylpiperazin-1-yl)acetyl cannabidiol (A7b)

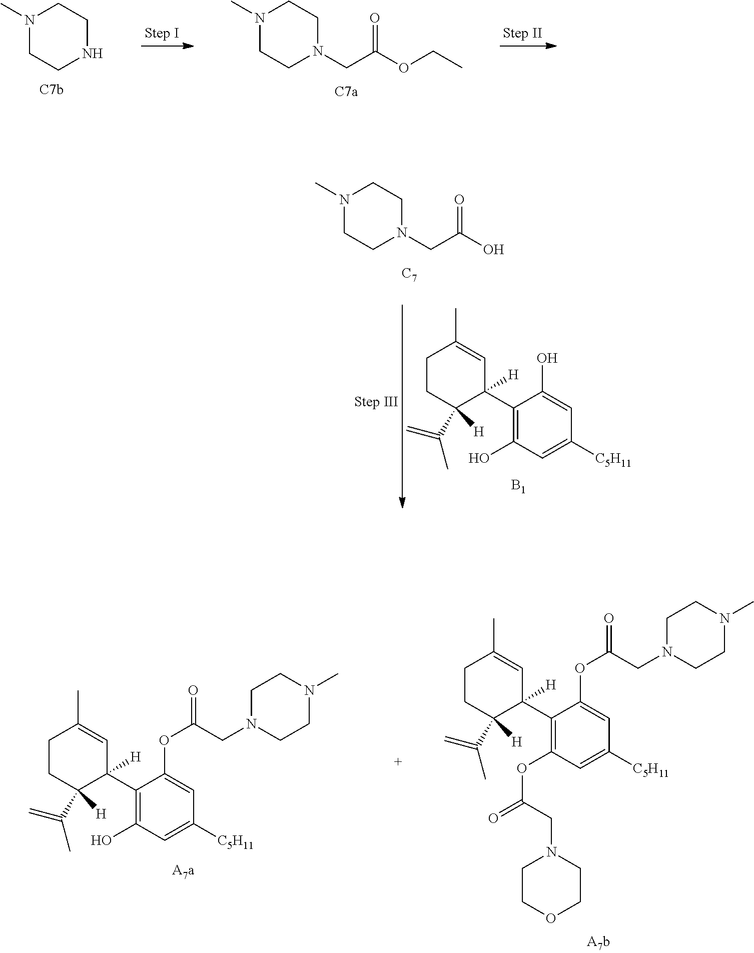

### Step 1: Synthesis of ethyl 2-(4-methylpiperazin-1-yl)acetate (C7a)

1-Methylpiperazine (C7b, 1 g, 1 eq.) and ethyl 2-chloroacetate (2.440 g, 2 eq.) and potassium carbonate (K₂CO₃) (4.140 g, 3 eq.) (act as base) were taken in RBF containing ACN solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected, dried over sodium sulphate and evaporated in vacuo. The crude material was purified by using silica gel chromatography eluted in mixture of methanol:chloroform (R_{f}˜0.5/5% methanol:chloroform) to get the pure compound (C7a, 0.909 g, 48%).

### Step 2: Synthesis of 2-(4-methylpiperazin-1-yl)acetic acid (C7)

Ethyl 2-(4-methylpiperazin-1-yl)acetate (C7a, 0.9 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography in mixture of methanol:chloroform (R_{f}˜0.5/10% methanol:chloroform) to get the pure product (C7, 0.7 g, 91%) as white solid compound. The pure compound was characterised by ¹H NMR, MS. ¹H NMR (500 MHz, MeOD) δ 3.26 (s, 2H), 2.88 (s, 4H), 2.79 (s, 4H), 2.55 (s, 3H); MS: (ESI+): m/z calcd for C₇H₁₅N₂O₂ 159.11; found 159.11.

### Step 3: Synthesis of CBD-Type Prodrug O-{2-(4-methylpiperazin-1-yl)acetyl cannabidiol (A7a) and O,O-{2-(4-methylpiperazin-1-yl)acetyl cannabidiol (A7b)

To a stirred solution of cannabidiol (B1, 200 mg, 0.636 mmol) and 2-(4-methylpiperazin-1-yl)acetic acid (C7, 202 mg, 1.272 mmol) in DCM/THF (1:1) and added DMAP (61 mg, 0.508 mmol), followed by 1-ethyl-3-(3-dimethylaminopropyl)carbodiimide (EDC) (485 mg, 2.544 mmol) under nitrogen atmosphere and stirred for 15 hours at 28° C. Reaction was monitored by TLC until reactant consumed. Two products (Mono and di-methylpiperazine CBD derivatives) were formed in the reaction at different R_{f} values i.e., R_{f}˜0.7/10% methanol:chloroform and R_{f}˜0.3/10% methanol:chloroform respectively. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. These compounds were separated/purified by using silica gel chromatography eluted in mixture of methanol:chloroform to get the mono-methylpiperazine CBD product i.e., O-{2-(4-methylpiperazin-1-yl)acetyl cannabidiol (A7a, 54 mg, 18%) and di-methylpiperazine CBD product i.e., O,O-{2-(4-methylpiperazin-1-yl)acetyl cannabidiol (A7b, 120 mg, 32%) as light yellow oil respectively.

These derivatives were characterized by ¹HNMR, ¹³C NMR, DEPT and HRMS. ¹H NMR (400 MHz, CDCl₃) δ 6.54 (s, 1H), 6.40 (s, 1H), 5.51 (s, 1H), 4.59 (s, 1H), 4.45 (s, 1H), 3.51-3.46 (m, 1H), 3.45-3.40 (m, 2H), 2.77-2.59 (m, 8H), 2.51-2.46 (m, 3H), 2.34 (s, 3H), 2.22-2.08 (m, 2H), 1.78-1.65 (m, 5H), 1.60 (s, 3H), 1.56-1.54 (m, 2H), 1.34-1.24 (m, 4H), 0.87 (t, J=6.6 Hz, 3H; ¹³C {¹H} NMR (101 MHz, CDCl₃) δ 170.79, 157.97, 151.40, 150.00, 143.27, 133.46, 127.28, 122.14, 115.50, 111.31, 59.83, 55.75, 53.34, 47.58, 45.91, 38.47, 36.61, 32.79, 32.06, 32.01, 30.84, 24.15, 23.80, 19.81, 14.63; [a]_{D}²⁰=−46 (c=1.0, MeOH); HRMS: (M+H)⁺: m/z calcd for C₂₈H₄₃N₂O₃ 455.3274; found 455.3274. ¹H NMR ¹H NMR (400 MHz, CDCl₃) δ 6.73 (s, 2H), 5.18 (s, 1H), 4.53 (s, 1H), 4.45 (s, 1H), 3.49-3.47 (m, 1H), 3.45-3.27 (m, 4H), 2.78-2.54 (m, 16H), 2.52-2.50 (m, 3H), 2.33 (s, 6H), 2.13-2.00 (m, 2H), 1.81-1.71 (m, 2H), 1.65 (s, 3H), 1.60-1.58 (m, 2H), 1.56 (s, 3H), 1.29-1.25 (m, 4H), 0.88 (t, J=6.8 Hz, 3H); ¹³C {¹H} NMR (101 MHz, CDCl₃) δ 70.42, 151.11, 149.37, 143.98, 134.52, 127.82, 125.77, 111.97, 59.69, 55.80, 53.42, 47.36, 46.03, 40.16, 36.32, 32.69, 31.99, 31.86, 30.44, 24.16, 23.79, 20.31, 14.63; [a]_{D}²⁰=−59 (c=1.0, MeOH); MS: (ESI+): m/z calcd for C₃₅H₅₅N₄O₄596.4223; found 596.4120.

### Example 8: Synthesis of CBD-Type Prodrug O-{2-(4-phenylpiperazin-1-yl)acetyl cannabidiol (A8a)

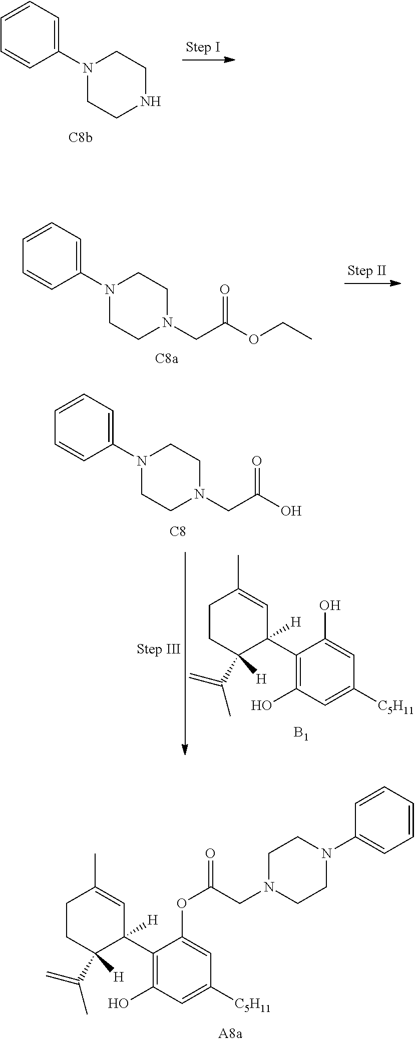

### Step 1: Synthesis of ethyl 2-(4-phenylpiperazin-1-yl)acetate (C8a)

1-Phenylpiperazine (C8b, 2 g, 1 eq.) and ethyl 2-chloroacetate (3.012 g, 2 eq.) and potassium carbonate (K₂CO₃) (5.110 g, 3 eq.) (act as base) were taken in RBF containing ACN solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected and dried over sodium sulphate and evaporated in vacuo. The crude material was purified by using silica gel chromatography eluted in mixture of ethyl acetate:hexane (R_{f}˜0.5/30% ethyl acetate:hexane) to get the pure compound (C8a, 2.340 g, 76%).

### Step 2: Synthesis of 2-(4-phenylpiperazin-1-yl)acetic acid (C8)

Ethyl 2-(4-phenylpiperazin-1-yl)acetate (C8a, 2.300 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography eluted in mixture of methanol:chloroform (R_{f}˜0.5/95% methanol:chloroform) to get the pure product (C8, 1.960 g, 96%) as white solid compound. The pure compound was characterised by ¹H NMR, MS. ¹H NMR (400 MHz, CDCl₃) δ 7.14 (t, J=7.2 Hz, 2H), 6.88 (d, J=8.0 Hz, 2H), 6.75 (t, J=7.6 Hz, 1H), 4.70 (s, 2H), 3.22-3.20 (m, 4H), 2.94-2.92 (m, 4H); MS: (ESI+): m/z calcd for C₁₂H₁₇N₂O₂ 221.12; found 221.35.

### Step 3: Synthesis of CBD-Type Prodrug O-{2-(4-phenylpiperazin-1-yl)acetyl cannabidiol (A8a)

To a stirred solution of cannabidiol (B1, 300 mg, 0.955 mmol) and 2-(4-phenylpiperazin-1-yl)acetic acid (C8, 315 mg, 1.432 mmol) in DCM/THF (1:1) and DMAP (93 mg, 0.286 mmol), followed by 1-ethyl-3-(3-dimethylaminopropyl)carbodiimide (EDC) (547 mg, 2.865 mmol) under nitrogen atmosphere and stirred for 15 hours at 27° C. Reaction was monitored by TLC until reactant consumed. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. This compound was separated/purified by using silica gel chromatography eluted in mixture of ethyl acetate:hexane (R_{f}˜0.5/25% ethyl acetate:hexane) to get the mono-phenylpiperazine CBD product i.e., O-{2-(4-phenylpiperazin-1-yl)acetyl cannabidiol (A8a, 330 mg, 67%) as light yellow oil.

This derivative was characterized by ¹H NMR, ¹³C NMR, DEPT and HRMS. ¹H NMR (400 MHz, CDCl₃) ¿ 7.31-7.25 (m, 2H), 7.00-6.84 (m, 3H), 6.56 (s, 1H), 6.44 (s, 1H), 6.00 (br s, OH), 5.54 (s, 1H),), 4.62 (s, 1H), 4.47 (s, 1H), 3.57-3.39 (m, 3H), 3.28 (t, J=4.5 Hz, 4H), 2.90-2.78 (m, 4H), 2.56-2.44 (m, 3H), 2.23-2.05 (m, 2H), 1.86-1.74 (m, 5H), 1.64-1.55 (m, 5H), 1.31-1.27 (m, 4H), 0.88 (t, J=6.5 Hz, 3H); ¹³C {¹H} NMR (101 MHz, CDCl₃) δ 170.87, 157.98, 153.04, 151.42, 150.10, 143.36, 130.43, 122.12, 121.53, 117.92, 115.63, 114.27, 111.34, 60.19, 54.44, 50.63, 47.39, 38.25, 36.61, 32.80, 32.07, 32.03, 30.84, 24.21, 23.81, 19.83, 14.64; [a]_{D}²⁰=−76 (c=1.0, MeOH); HRMS: (M+H)⁺: m/z calcd for C₃₃H₄₅N₂O₃517.3430; found 517.3430.

### Example 9: Synthesis of CBD-Type Prodrug O-{2-(piperidin-1-yl)acetyl} cannabidiol (A9a)

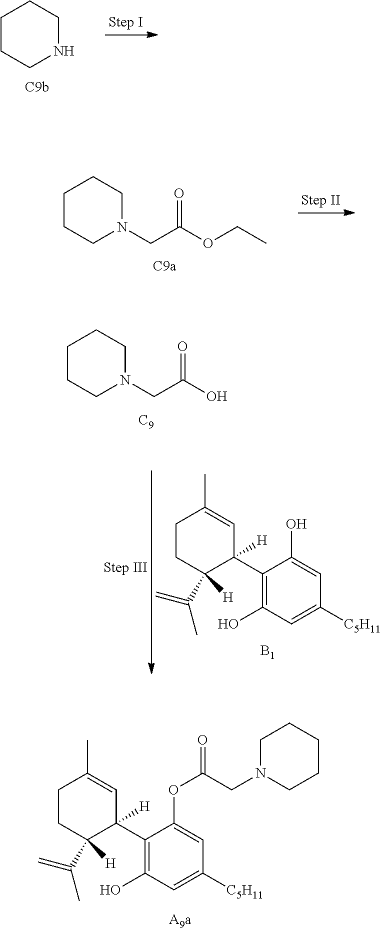

### Step 1: Synthesis of ethyl 2-(piperidin-1-yl)acetate (C9a)

Piperidine (2 g, 1 eq.) (C9b, 2 g, 1 eq.) and ethyl 2-chloroacetate (5.741 g, 2 eq.) and potassium carbonate (K₂CO₃) (9.741 g, 3 eq.) (act as base) were taken in RBF containing ACN solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected, dried over sodium sulphate and evaporated in vacuo. The crude material was purified by using silica gel chromatography eluted in mixture of ethyl acetate:hexane (R_{f}˜0.5/20% ethyl acetate:hexane) to get the pure compound (C9a, 2.8 g, 69%).

### Step 2: Synthesis of 2-(piperidin-1-yl)acetic acid (C9)

Ethyl 2-(piperidin-1-yl)acetate (C9a, 2.8 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography eluted in mixture of ethyl acetate:hexane (R_{f}˜0.5/50% ethyl acetate:hexane) to get the pure product (C9, 2.1 g, 89%) as yellow solid compound. The pure compound was characterised by ¹H NMR, MS. ¹H NMR (400 MHz, MeOD) δ 3.49 (s, 2H), 3.20-3.19 (m, 4H), 1.80 (s, 4H), 1.58 (s, 2H); MS: (ESI+): m/z calcd for C₇H₁₄NO₂ 144.10; found 144.10.

### Step 3: Synthesis of CBD-Type Prodrug O-{2-(piperidin-1-yl)acetyl} cannabidiol (A9a)

To a stirred solution of cannabidiol (B1, 300 mg, 0.955 mmol) and 2-(piperidin-1-yl)acetic acid (C9, 181 mg, 1.272 mmol) in DCM/THF (1:1), and added DMAP (61 mg, 0.508 mmol), followed by 1-ethyl-3-(3-dimethylaminopropyl)carbodiimide (EDC) (485 mg, 2.544 mmol) under nitrogen atmosphere and stirred for 16 hours at 26° C. Reaction was monitored by TLC until reactant consumed. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. This compound was separated/purified by using silica gel chromatography eluted in mixture of ethyl acetate:hexane (R_{f}˜0.5/20% ethyl acetate:hexane) to get the mono-piperidine CBD product ie., O-{2-(piperidin-1-yl)acetyl} cannabidiol (A9a, 80 mg, 28%) as light yellow oil. This derivative was characterized by ¹HNMR, ¹³C NMR, DEPT and HRMS.

¹H NMR (400 MHz, CDCl₃) δ 6.54 (s, 1H), 6.41 (s, 1H), 5.52 (s, 1H), 4.59 (s, 1H), 4.44 (s, 1H), 3.57-3.46 (m, 1H), 3.41-3.31 (m, 2H), 2.67-2.55 (m, 4H), 2.53-2.40 (m, 3H), 2.21-2.00 (m, 2H), 1.83-1.70 (m, 5H), 1.67-1.62 (m, 4H), 1.61 (s, 3H), 1.58-1.52 (m, 2H), 1.49-1.43 (m, 2H), 1.32-1.25 (m, 4H), 0.87 (t, J=6.7 Hz, 3H); ¹³C {¹H} NMR (101 MHz, CDCl₃) δ 170.88, 157.91, 151.49, 150.11, 143.12, 133.51, 127.80, 122.31, 115.68, 114.17, 111.43, 60.95, 55.77, 55.67, 55.62, 46.66, 37.77, 36.62, 32.83, 32.14, 32.00 30.84, 26.74, 25.06, 24.21, 23.85, 19.74, 14.68. [a]_{D}²⁰=−56 (c=1.0, MeOH); HRMS: (M+H)⁺: m/z calcd for C₂₈H₄₂NO₃ 440.3165; found 440.3165.

### Example 10: Synthesis of CBD-Type Prodrug O-{2-(pyrrolidin-1-yl)acetyl} cannabidiol (A10a)

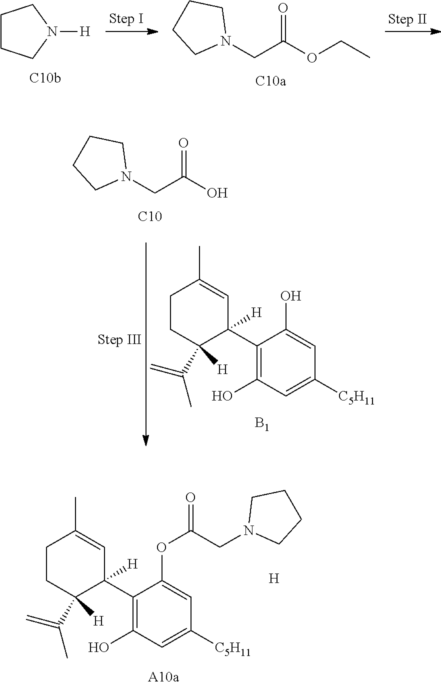

### Step 1: Synthesis of ethyl 2-(pyrrolidin-1-yl)acetate (C10a)

Pyrrolidine (C10b, 1.5 g, 1 eq.) and ethyl 2-chloroacetate (5.154 g, 2 eq.) and potassium carbonate (K₂CO₃) (8.746 g, 3 eq.) (act as base) were taken in RBF containing DMF solvent and stirred for 5 hours at 30° C. until completion as monitored by TLC. After reaction was completed, solvent evaporated through rota-evaporator. The concentrated reaction mixture was diluted with ethyl acetate and washed with sodium bicarbonate solution. The organic and aqueous layers were separated. Aqueous layer was washed 2 or 3 more times with ethyl acetate. Organic layer was collected, dried over sodium sulphate and evaporated in vacuo. The crude material was purified by using silica gel chromatography eluted in mixture of methanol:chloroform (R_{f}˜0.5/10% methanol:chloroform) to get the pure compound (C10a, 1.9 g, 85%).

### Step 2: Synthesis of 2-(pyrrolidin-1-yl)acetic acid (C10)

Ethyl 2-(pyrrolidin-1-yl)acetate (C10a, 1.9 g, 1 eq.) and 2N NaOH (act as base) were taken in RBF containing MeOH solvent and stirred for 10 minutes at 25° C. until completion as monitored by TLC. Filtered the reaction mixture and evaporated the solvent through rota-evaporator. Purification was done by using silica gel chromatography eluted in mixture of methanol:chloroform (R_{f}˜0.5/95% methanol:chloroform) to get the pure product (C10, 1.401 g, 89%) as yellow semi-solid compound. The pure compound was characterised by ¹H NMR, MS. 1H NMR (400 MHz, MeOD) δ 3.57 (s, 2H), 3.27-3.18 (m, 2H), 2.09-1.87 (m, 4H); MS: (ESI+): m/z calcd for C₆H₁₂NO₂ 130.08; found 130.15.

### Step 3: Synthesis of CBD-Type Prodrug O-{2-(pyrrolidin-1-yl)acetyl} cannabidiol (A10a)

To a stirred solution of cannabidiol (B1, 300 mg, 0.955 mmol) and 2-(pyrrolidin-1-yl)acetic acid (C10, 164 mg, 1.272 mmol) in DCM/THF (1:1) and added DMAP (61 mg, 0.508 mmol) followed by 1-ethyl-3-(3-dimethylaminopropyl)carbodiimide (EDC) (486 mg, 2.544 mmol) under nitrogen atmosphere and stirred for 16 hours at 26° C. Reaction was monitored by TLC until reactant consumed. Reaction mixture was dried in vacuo. The concentrated reaction mixture was workup with ethyl acetate and sodium bicarbonate solution. Aqueous layer was washed 2 or 3 more times with ethyl acetate. Organic layer was compiled, dried over sodium sulphate and evaporated in vacuo. This compound was separated/purified by using silica gel chromatography eluted in mixture of ethyl acetate:hexane (TLC R_{f}˜0.5/30% ethyl acetate:hexane) to get the mono-pyrrolidine CBD product ie., O-{2-(pyrrolidin-1-yl)acetyl} cannabidiol (A10, 51 mg, 18.8%) as light yellow oil.

This derivative was characterized by ¹HNMR, ¹³C NMR, DEPT and HRMS. ¹H NMR (400 MHz, CDCl₃) δ 6.55 (s, 1H), 6.42 (s, 1H), 6.00 (s, 1H), 5.54 (s, 1H), 4.60 (s, 1H), 4.44 (s, 1H), 3.60-3.46 (m, 3H), 2.81-2.66 (m, 4H), 2.54-2.40 (m, 3H), 2.23-2.04 (m, 2H), 1.85-1.83 (m, 4H), 1.77 (s, 3H), 1.70-1.64 (m, 2H), 1.60 (s, 3H), 1.59-1.54 (m, 2H), 1.31-1.25 (m, 4H), 0.87 (t, J=6.9 Hz, 3H); ¹³C {¹H} NMR (101 MHz, CDCl₃) δ 171.00, 158.01, 151.39, 149.96, 143.30, 134.34, 127.69, 122.09, 116.23, 113.94, 111.39, 57.62, 55.25, 55.21, 55.17, 46.61, 37.74, 36.63, 32.84, 32.18, 31.98, 30.80, 24.84, 24.30, 23.87, 19.80, 14.71; [a]_{D}²⁰=−63 (c=1.0, MeOH); HRMS: (M+H) m/z calcd for C₂₇H₄₀NO₃ 426.3008; found 426.3008.

### Example 11: Ex Vivo Efficacy Studies

### Buffer Preparation

For the preparation of citrate buffer solution (pH=7.6), 100 ml of HPLC water, 0.25 M sucrose and 40 mM sodium citrate were taken.

### Calibration Curve for CBD Based Prodrug and Cannabidiol (B1)

To construct a calibration curve, five different concentration such as 0.78, 1.56, 3.12, 6.25, 12.5, 25, 50 mg/ml of CBD based prodrugs and the parent cannabidiol were prepared. All sample were analyzed by 4.6×250 mm, 5 μm end capped RP-C18 column and the mobile phase containing buffer (0.1% formic acid in water and 0.1% formic acid in acetonitrile) and in some case methanol were used at the flow rate of 1 mL/min at column temperature of 25° C. at 210 nm, 190 nm, 254 nm wavelength range. A volume of 10 μL was injected and a total run time of the assay range was between 15-40 mins. The markers were estimated by HPLC using gradient technique.

### Preparation of Plasma Sample

For the preparation of plasma sample, fresh 20 ml of human blood was taken to which 2 ml of citrate buffer was added and then vortex it for about 5 minutes. Immediately after this, the mixture was centrifuged at 8000 rpm for 20 minutes, resulting in the separation of two-layer like RBC and plasma (ethical approval number 1EC/GMC/Cat A/2020/267).

### General Procedure for Ex Vivo Human Plasma Stability Studies

### Sample Preparation

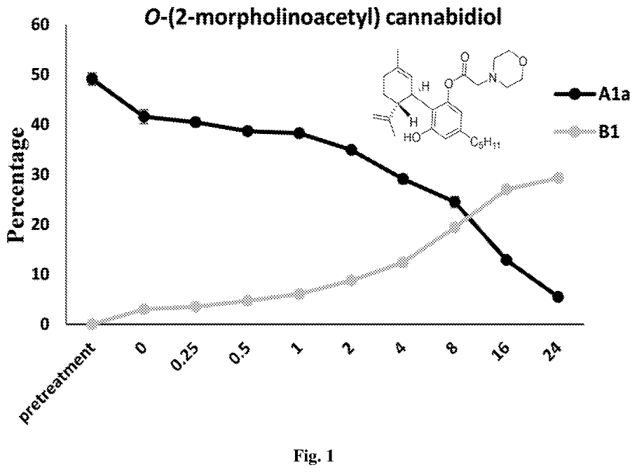

To check the stability of prodrugs in plasma, stock solution of prodrug in DMSO (5 mg/2 ml) was prepared and placed at −20° C. The plasma sample of 50 μl was taken in different eppendorf's followed by the addition of 5 μl prodrug solution, vertex for 2 min., incubated at 37° C. at the different time point (Schedule provided in Table 1). After 24 h, all the sample were added with 200 μl MeOH with the help of vertex for about 2 minutes followed by centrifuge for about 10 minutes with 4000 rpm and two layers of sample were separated. The upper layer of methanol sample was taken and immediately the methanol sample was analyzed by 4.6×250 mm, 5 μm end capped RP-C₁₈ column and the mobile phase containing buffer (0.1% formic acid in water and 0.1% formic acid in acetonitrile) and in some case methanol were used at the flow rate of 1 mL/min at column temperature of 25° C. at 210 nm, 190 nm, 254 nm wavelength range. A volume of 10 μL was injected and a total run time of the assay range was 15-40 mins. The markers were estimated by HPLC using gradient technique. The results are depicted in FIG. 1 to FIG. 10.

TABLE 1
Time points for drug addition of plasma in incubator

| Time points for drug | | | |
|---|---|---|---|
| addition of plasma in | | | Similar time |
| incubator | Time point | | (Lifting point) |
| 2 PM DAY 2 | 0 | hour | 2 PM DAY 2 |
| 1:45 PM DAY 2 | 0.25 | hour | 2 PM DAY 2 |
| 1:30 PM DAY 2 | 0.5 | hour | 2 PM DAY 2 |
| 1 PM DAY 2 | 1 | hour | 2 PM DAY 2 |
| 12 PM DAY 2 | 2 | hours | 2 PM DAY 2 |
| 10 AM DAY 2 | 4 | hours | 2 PM DAY 2 |
| 6 AM DAY 2 | 8 | hours | 2 PM DAY 2 |
| 10 PM DAY 1 | 16 | hours | 2 PM DAY 2 |
| 2 PM DAY 1 | 24 | hours | 2 PM DAY 2 |

FIG. 1 shows the release of cannabidiol (B1) from prodrug, O-(2-morpholinoacetyl) cannabidiol (A1a) in ex vivo assay system. The sample was treatment with blood plasma having a citrate buffer solution and the analysis was performed as per time points mentioned in the general procedure of ex-vivo studies (Example 11). The results revealed that the prodrug, O-(2-morpholinoacetyl) cannabidiol (A1a) released the cannabidiol (B1) from the 0% to 29.3% over period of time. At the initial time point, the concentration of O-(2-morpholinoacetyl) cannabidiol (A1a) found to be 49.1%, however, after 24 h, 5.5% of prodrug left. The quantification results were reported as per procedure mentioned in the general procedure.

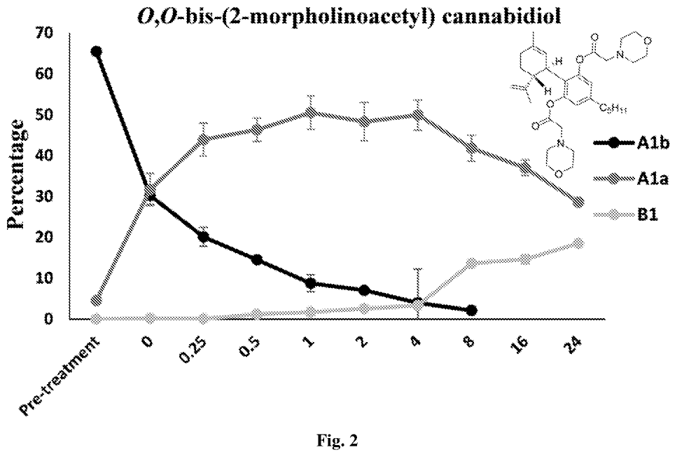

FIG. 2 shows the release of cannabidiol (B1) from prodrug, O,O-(2-morpholinoacetyl) cannabidiol (A1b) in ex vivo assay system. The sample was treatment with blood plasma having a citrate buffer solution and the analysis was performed as per general procedure mentioned above in Example 11. The results revealed that the prodrug, O,O-(2-morpholinoacetyl) cannabidiol (A1b) immediately converted into mono substituted CBD derivative i.e., O-(2-morpholinoacetyl) cannabidiol (A1a) and then released the cannabidiol B1 over a period of time and the concentration of 18.5% reached at the end of 24 h. The quantification results were reported as per procedure mentioned in the general procedure.

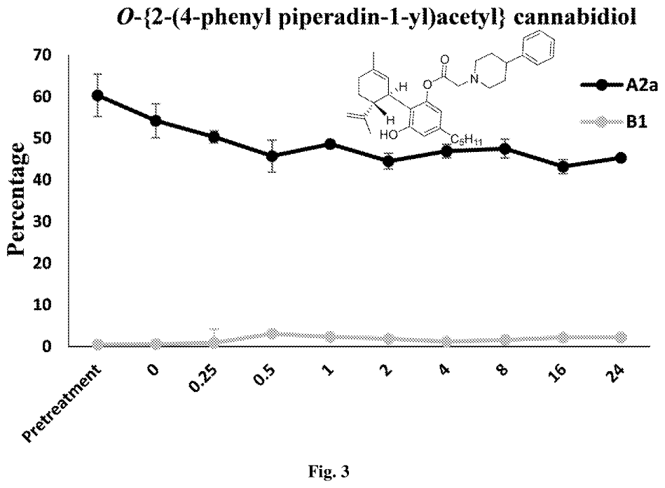

FIG. 3 shows the release of cannabidiol (B1) from prodrug, O-{2-(4-phenyl piperadin-1-yl)acetyl} cannabidiol (A2a) in ex vivo assay system. The sample was treatment with blood plasma having a citrate buffer solution and the analysis was performed as per general procedure mentioned above in Example 11. The results observed that, prodrug, O-{2-(4-phenyl piperadin-1-yl)acetyl} cannabidiol (A2a) is quite stable, however some release was observed over a period of 24 h and the maximum concentration of cannabidiol (B1) was observed as 2.2% after 24 h. The quantification results were reported as per procedure mentioned in the general procedure.

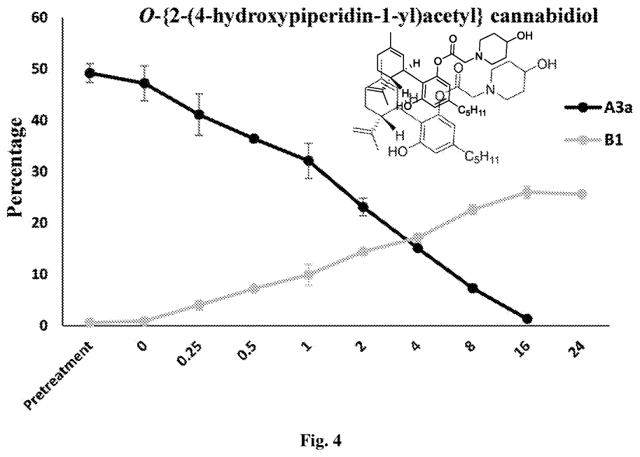

FIG. 4 shows the release of cannabidiol (B1) from prodrug, O-{2-(4-hydroxypiperidin-1-yl)acetyl} cannabidiol (A3a) in ex vivo assay system. The analysis was performed as per general procedure mentioned above in Example 11. The results revealed that, the concentration of the prodrug, O-{2-(4-hydroxypiperidin-1-yl)acetyl} cannabidiol (A3a) decreased from the concentration 49.2% (Pre-treatment) to below detection limit after 24 h. The concentration of CBD (B1) was gradually converted increased and reaches the value of 25.6% at 24 h. The quantification results were reported as per procedure mentioned in the general procedure.

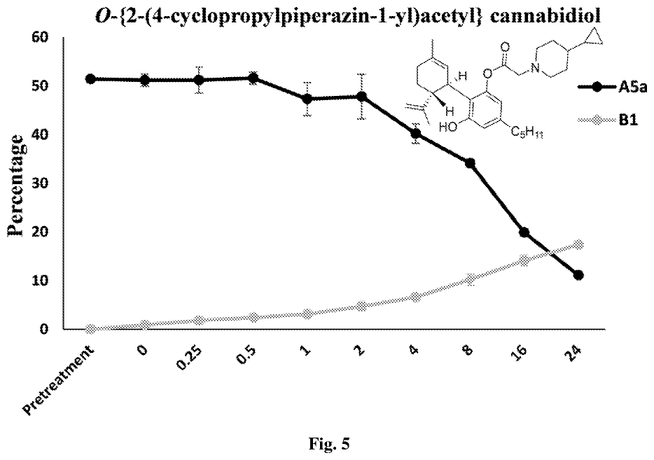

FIG. 5 shows the release of cannabidiol (B1) from prodrug, O-{2-(4-cyclopropylpiperazin-1-yl)acetyl} cannabidiol (A5a) in ex vivo assay system. The sample was treatment with blood plasma having a citrate buffer solution and the analysis was performed as per general procedure mentioned above in Example 11. The results revealed that, the concentration of the prodrug, O-{2-(4-cyclopropylpiperazin-1-yl)acetyl} cannabidiol (A5a) decreased over the period of time and concentration of cannabidiol (B1) was gradual increase from the 0% to 17.4%. At the initial time point, the concentration of O-{2-(4-cyclopropylpiperazin-1-yl)acetyl} cannabidiol (A5a) found to be 51.4% (Pre-treatment), however, after 24 h, 11.1% of prodrug left. The quantification results were reported as per procedure mentioned in the general procedure.

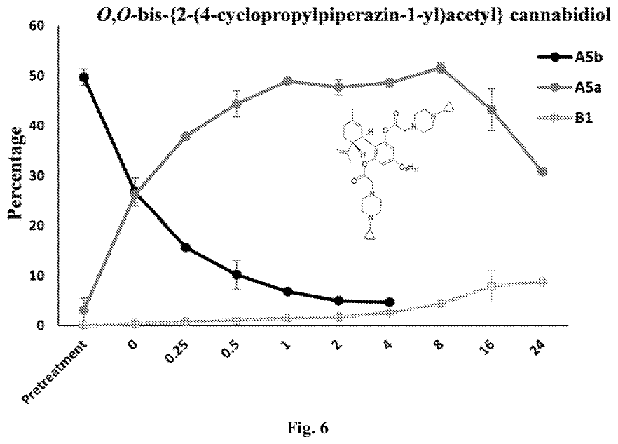

FIG. 6 shows the release of cannabidiol (B1) from prodrug O,O-{2-(4-cyclopropylpiperazin-1-yl)acetyl} cannabidiol (A5b) in ex vivo assay system. The sample was treatment with blood plasma having a citrate buffer solution and the analysis was performed as per general procedure mentioned above in Example 11. The results revealed that the prodrug, O,O-{2-(4-cyclopropylpiperazin-1-yl)acetyl} cannabidiol (A5b) immediately converted into mono substituted CBD derivative i.e., O-{2-(4-cyclopropylpiperazin-1-yl)acetyl} cannabidiol (A5a), however, its further hydrolysis is quite slow and release started after 16 h. The concentration of CBD (B1) was observed as 8.8% after 24 h. The quantification results were reported as per procedure mentioned in the general procedure.

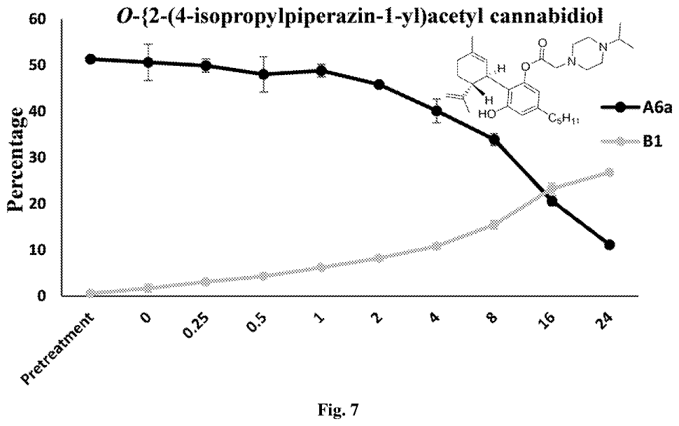

FIG. 7 shows the release of cannabidiol (B1) from prodrug, O-{2-(4-isopropylpiperazin-1-yl)acetyl cannabidiol (A6a) in ex vivo assay system. The sample was treatment with blood plasma having a citrate buffer solution and the analysis was performed as per general procedure mentioned above in Example 11. The results revealed that, the concentration of the prodrug, O-{2-(4-isopropylpiperazin-1-yl)acetyl cannabidiol (A6a) decreased over the period of time and by gradual increase the concentration of cannabidiol (B1) from the 0.6% to 26.8%. At the initial time point, the concentration of O-{2-(4-isopropylpiperazin-1-yl)acetyl cannabidiol (A6a) found to be 51.3% (Pre-treatment), however, after 24 h, 11.1% of prodrug left. The quantification results were reported as per procedure mentioned in the general procedure.

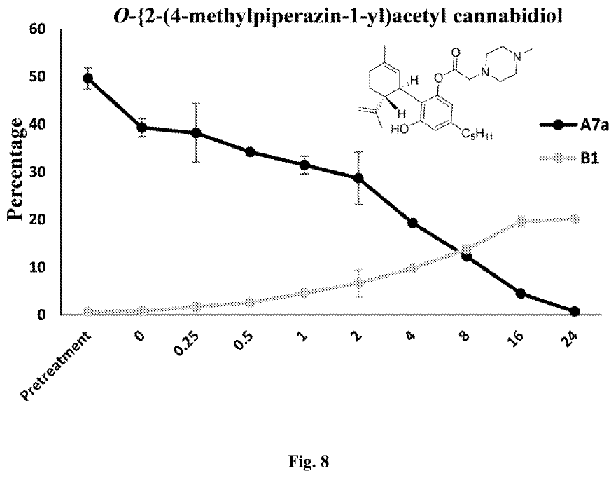

a. FIG. 8 shows the release of cannabidiol (B1) from prodrug, O-{2-(4-methylpiperazin-1-yl)acetyl cannabidiol (A7a) in ex vivo assay system. The sample was treatment with blood plasma having a citrate buffer solution and the analysis was performed as per general procedure mentioned above in Example 11. The results revealed that, the concentration of the prodrug, O-{2-(4-methylpiperazin-1-yl)acetyl cannabidiol (A7a) decreased over the period of time and by gradual increase the concentration of cannabidiol (B1) from the 0.6% to 20.1%. At the initial time point, the concentration of O-{2-(4-methylpiperazin-1-yl)acetyl cannabidiol (A7a) found to be 49.6% (Pre-treatment), however, after 24 h, 0.7% of prodrug left. The quantification results were reported as per procedure mentioned in the general procedure.

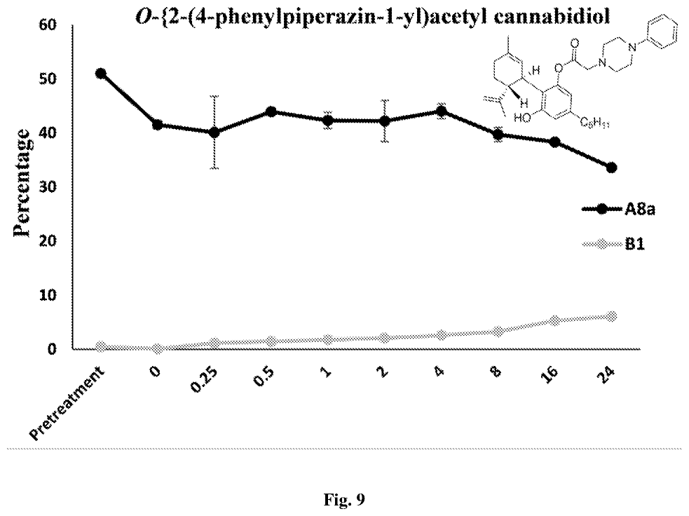

FIG. 9 shows the release of cannabidiol (B1) from prodrug, O-{2-(4-phenylpiperazin-1-yl)acetyl cannabidiol (A8a) in ex vivo assay system. The sample was treatment with blood plasma having a citrate buffer solution and the analysis was performed as per general procedure mentioned above in Example 11. The results revealed the concentration of the prodrug, O-{2-(4-phenylpiperazin-1-yl)acetyl cannabidiol (A8a) is quite stable, however some release was observed over a period of 24 h and the maximum concentration of cannabidiol (B1) was observed as 6.1% after 24 h. The quantification results were reported as per procedure mentioned in the general procedure.

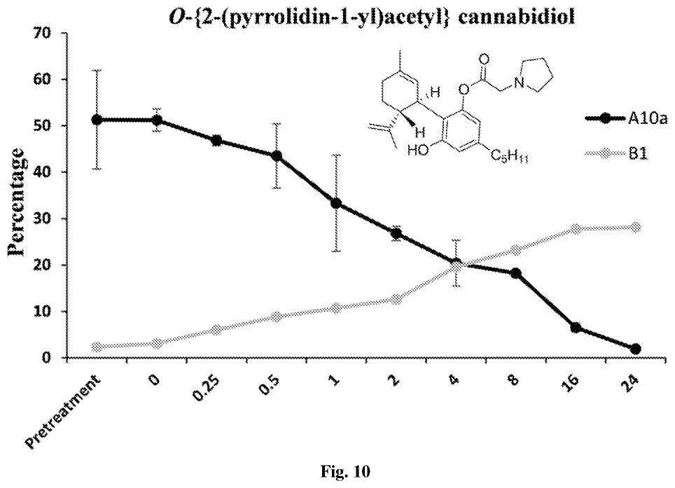

FIG. 10 shows the release of cannabidiol (B1) from prodrug, O-{2-(pyrrolidin-1-yl)acetyl} cannabidiol (A10a) in ex vivo assay system. The sample was treatment with blood plasma having a citrate buffer solution and the analysis was performed as per general procedure mentioned above in Example 11. The results revealed that, the concentration of the prodrug, O-{2-(pyrrolidin-1-yl)acetyl} cannabidiol (A10a) decreased over the period of time and gradual increase in the concentration of cannabidiol (B1) was observed with value ranging from 2.3% to 28.1% after 24 h. At the initial time point, the concentration of O-{2-(pyrrolidin-1-yl)acetyl} cannabidiol (A10a) found to be 51.3% (Pre-treatment), however, after 24 h, 1.9% of prodrug left. The quantification results were reported as per procedure mentioned in the general procedure.

### Example 12: Comparative Pharmacokinetics Studies in Mice

### Methodology

Comparative pharmacokinetic studies of A1b and B1 were performed in healthy male Swiss mice. Animals were maintained under standard laboratory conditions and given the normal pellet diet with free access to water. Necessary approval was obtained for animal experimentations from our institute's Institutional Animal Ethics Committee. On the day of the experiment, animals were randomly arranged into three subgroups, with five mice per sub-group for each pharmacokinetic study. The experimental dose of B1 was at 75 mg/kg or its equivalent for A1b. Corn oil was used as a vehicle for dose preparation at the dose-volume of 10 mL/kg. After oral dosing, blood sampling was done using sparse sampling strategy at 0 h, 0.25 h, 0.5 h, 1 h, 2 h, 4 h, 8 h, 12 h, and 24 h into microcentrifuge tubes containing anticoagulant. Each blood sample was centrifuged at 8000 rpm for 10 min to obtain 50 μL of plasma. Sample processing was done by adding acetonitrile (200 μL) containing diazepam (25 ng/mL) as internal standard (IS). After that, the sample was vortex mixed for 2 min, centrifuged at 14000 rpm for 10 min, decanted into inner vials. Quantification of B1 was done by LC-MS/MS [Model: TSQ Endura (MS) & Ultimate 3000 (HPLC); Make: Thermo Scientific] using a matrix match calibration curve (3.9 to 1000 ng/mL). Then, plasma concentrations data were used to calculate various pharmacokinetic parameters by the non-compartmental method using PK solution software.

### Result

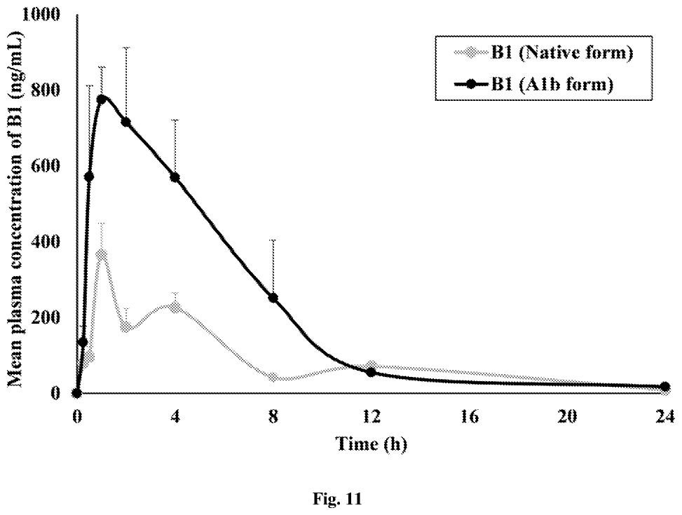

Comparative pharmacokinetic studies of A1b and B1 were carried out upon oral administration using a mice model. The mean plasma concentration versus time profiles of B1 and its calculated average pharmacokinetic parameters based on the dose of B1 are represented in FIG. 11 and Table 2 and 3. Overall plasma exposure of B1 was substantially augmented by 2.5-fold upon oral administration of A1b compared to its native form (B1). The obtained results are associated with comparatively enhanced maximum plasma concentration of B1 after oral dosing of A1b in comparison to its as such form (B1). Moreover, slower elimination with a higher half-life of B1 was observed after oral administration of A1b compared to its native form (B1). Results demonstrate the suitability of a prodrug approach for better oral absorption of CS-01.

TABLE 2
Mean plasma concentration of CS-01 after oral administration of O,O-
bis-(2-morpholinoacetyl) cannabidiol (A1b) or cannabidiol (B1) in mice.

| Compound | | | | | | | | | |
|---|---|---|---|---|---|---|---|---|---|
| for oral | Mean plasma concentration(ng/mL) of B1 | | | | | | | | |
| dosing | 0 h | 0.25 h | 0.5 h | 1 h | 2 h | 4 h | 8 h | 12 h | 24 h |
| B1 | 0 | 85 ± 7 | 96 ± 27 | 308 ± 87 | 178 ± 45 | 226 ± 39 | 36 ± 8 | 71 ± 8 | 9 ± 1 |
| A1b | 0 | 135 ± 42 | 571 ± 240 | 775 ± 85 | 715 ± 196 | 569 ± 152 | 251 ± 153 | 55 ± 8 | 17 ± 3 |

TABLE 3
Pharmacokinetic parameters of B1 after oral administration
of O,O-bis-(2-morpholinoacetyl) cannabidiol
(A1b) or cannabidiol (B1) in mice.

| | |
|---|---|
| Pharmacokinetic | B1 |

| parameter | Native form | A1b form |
|---|---|---|
| Cₘₐₓ (ng/mL) | 366 | 775 |
| Tₘₐₓ (h) | 1.0 | 1.0 |
| T_{1/2} (h) | 4.0 | 7.0 |
| AUC₀₋ₜ (ng · h/mL) | 2060 | 5148 |
| AUC_{0-∞} (ng · h/mL) | 2112 | 5315 |
| V_{d} (L/kg) | 206 | 142 |
| Cl (L/h/kg) | 36 | 14 |
| MRT (h) | 6.9 | 8.4 |

### Advantages of the Present Invention

The present invention deals the synthesis of prodrug of CBD-type phytocannabinoids:

1. To enhance the plasma stability of the phytocannabinoids by reducing the degradation of CBD in gastro-intestinal (GI) track.

2. To enhance the bioavailability of phytocannabinoids by increase the aqueous solubility.

3. To help in the slow release of the phytocannabinoids over longer period of time.

4. To help in the reduction of the phytocannabinoids dosage in the finished product.

1-11. (canceled)

12. A prodrug of cannabidiol, the prodrug comprising a compound of formula (A): 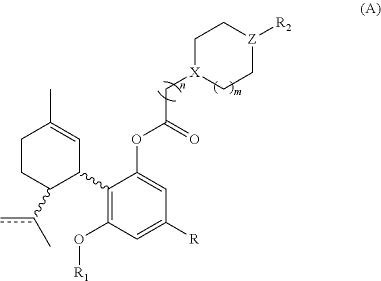
where:
R and R₂ are independently selected from the group consisting of H, OH, protected hydroxyl, C₁-C₁₄ alkyl, C₁-C₁₄ alkenyl, C₁-C₁₄ alkynyl, C₁-C₁₄ acyl, aryl, heteroaryl, cycloalkyl, and heterocycle, where:
alkyl, alkenyl, alkynyl, or acyl are optionally substituted with one or more groups each independently selected from the group consisting of halogen, —OH, alkyl, —O-alkyl, NR′R″, S-alkyl, —SO-alkyl, —SO₂-alkyl, S-aryl, —SO-aryl, —SO₂-aryl, —SO₂—N-aryl, —N—SO₂, -arylalkenyl, alkynyl, unsubstituted aryl, unsubstituted heteroaryl, cycloalkyl or heterocycle, substituted aryl, and substituted heteroaryl, where the substituted aryl and the substituted heteroaryl are substituted with one or more substituents independently selected from the group consisting of halogen, OH, alkyl, —O-alkyl, —COOH, —C(O), —C alkyl, —C(O)OC, alkyl, and NR′R″;
R′ and R″ are independently selected from the group consisting of H, C₁-C₁₀ alkyl, C₁-C₁₀ alkenyl, C₁-C₁₀ alkynyl, and C₁-C₁₀ acyl;
X is C or N;
Z is C, N, or O;
n is an integer from 1 to 10;
m is 0, 1, or 2;
R₁ is selected from —H or a moiety of formula (A1): 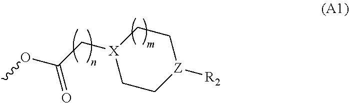
where R2, X, Z, m, and n are as defined in formula (A); [symbol]
represents a single bond or a double bond; [symbol]
represents a single bond of any stereochemistry,
wherein the prodrug has a mean plasma concentration from 17 ng/ml to 775 ng/mL.

13. The prodrug according to claim 12, wherein the compound comprises two stereocenters each independently having R symmetry or S symmetry.

14. The prodrug according to claim 12, wherein the compound is selected from the group consisting of

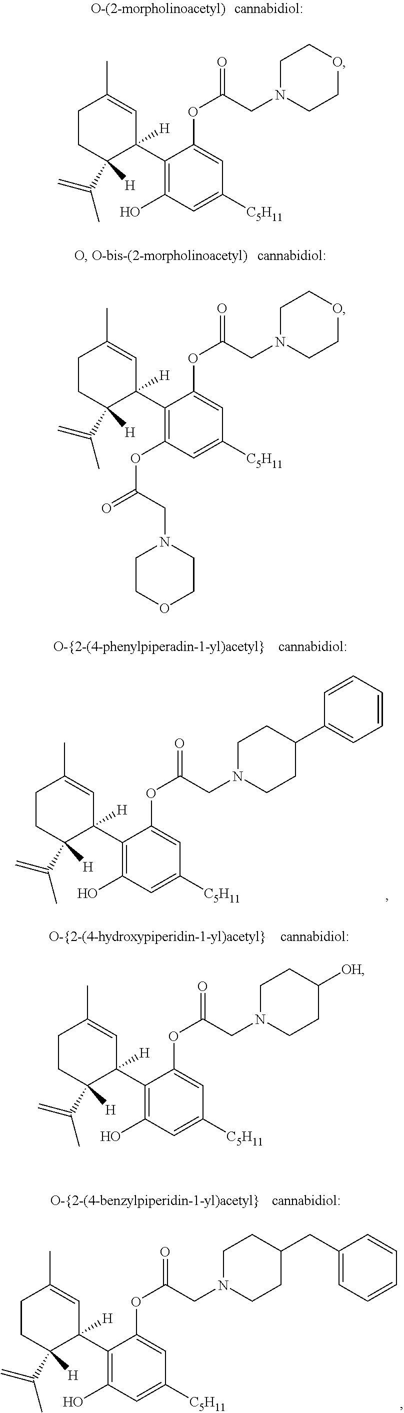

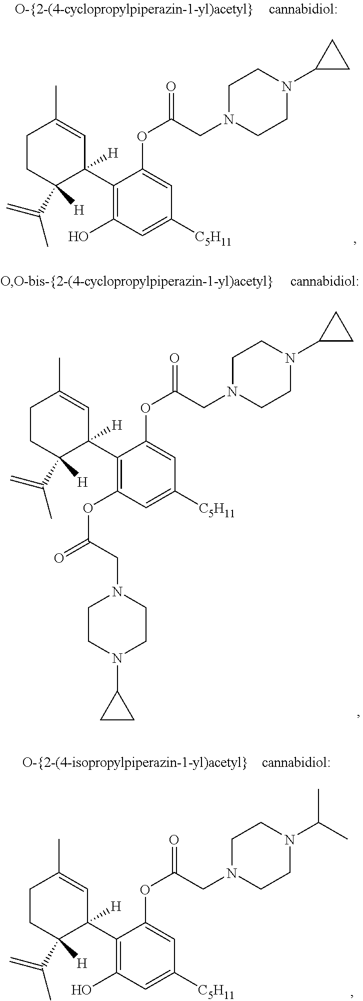

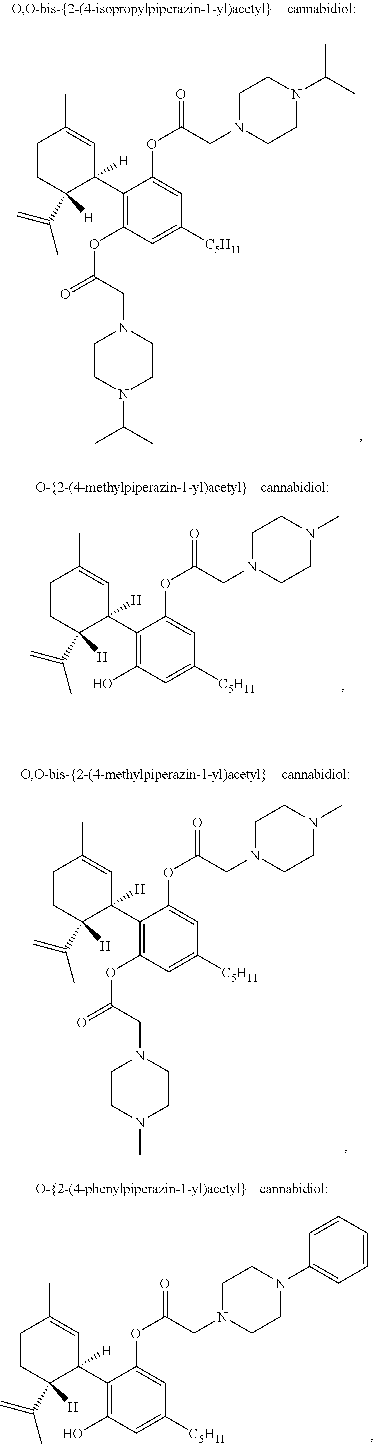

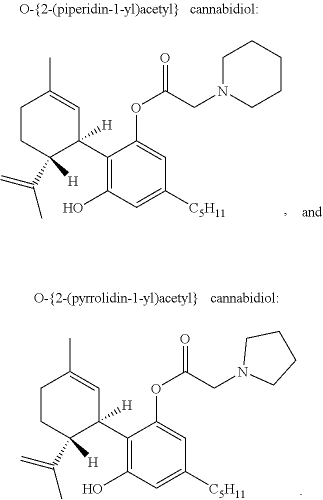

15. A process preparing the prodrug according to claim 12, the process comprising:
(i) reacting, in the presence of a coupling agent, a compound of formula (B): 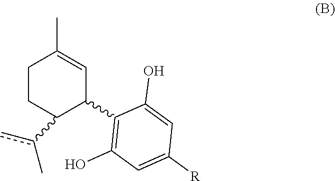
where R is as defined in formula (A),
and a compound of formula (C): 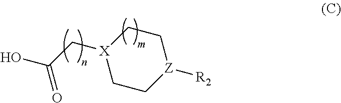
where R₂, m, and n are as defined in formula (A),
to obtain a reaction mixture; and
(ii) treating the reaction mixture obtained in (i) with a base in the presence of a dry solvent at from 25° C. to 30° C. for 13 hours to 16 hours to obtain the compound of Formula (A).

16. The process according to claim 15, wherein the compound of Formula (C) is selected from the group consisting of compound (C1), compound (C2), compound (C3), compound (C4), compound (C5), compound (C6), compound (C7), compound (C8), compound (C9), and compound (C10):

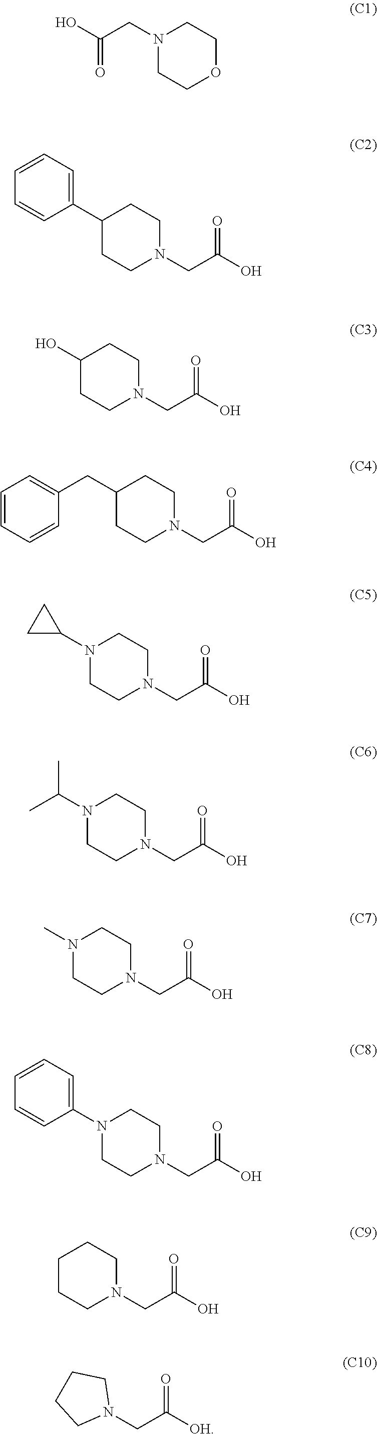

17. The process according to claim 15, wherein the coupling agent is selected from the group consisting of N,N-dicyclohexylcarbodiimide, a mixture of N,N-diisopropylcarbodiimide and carbonyldiimidazole, and 1-ethyl-3-(3-dimethylaminopropyl)carbodiimide.

18. The process according to claim 15, wherein the base is selected from the group consisting of diethylamino pyridine, 2,6-lutidine, trimethylamine, and diethylamino pyridine.

19. The process according to claim 15, wherein the solvent is selected from the group consisting of dichloromethane, chloroform, isopropanol, acetone, acetonitrile, and combinations thereof.

20. A pharmaceutical composition comprising:
the prodrug according to claim 12,
corn oil,
dimethylsulfoxide, and
methanol.

21. A method for enhancing a bioavailability of the prodrug according to claim 12, wherein the method comprises administering the prodrug to the subject.

22. A method for treating a disease or disorder in a subject having the disease or disorder, the method comprising administering the prodrug according to claim 12 to the subject.
